SEQUENCE LISTING (6 sequences)

SEQ ID NO: 1 - DNA, 659 bp, Lactobacillus spp.
tggaaacagatgctaataccgcataacaacaaaagccacatggcttttgtttgaaagatggctttagctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggccacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtcc

SEQ ID NO: 2 - DNA, 22 bp, Lactobacillus spp.
tggaaacagatgctaataccgc

SEQ ID NO: 3 - DNA, 20 bp, Lactobacillus spp.
attagataccctggtagtcc

SEQ ID NO: 4 - DNA, 19 bp, Lactobacillus spp.
agcagtagggaatcttcca

SEQ ID NO: 5 - DNA, 18 bp, Lactobacillus ssp.
attycaccgctacacatg

SEQ ID NO: 6 - DNA, 22 bp, Lactobacillus ssp.
acaatggacgaaagtctgagtg

(19) United States
(12) Patent Application Publication
(10) Pub. No.: US 2016/0032413 A1
(43) Pub. Date: Feb. 4, 2016

(54) NANOPRODUCT COMPRISING LACTOBACILLUS REUTERI DAN080 USEFUL IN PROPHYLAXIS AND MEDICINE, BOTH HUMAN AND VETERINARY AND MEDICAL USE OF THE SAME

(71) Applicant: Danuta Kruszewska, Warszawa (PL)

(72) Inventor: Danuta Kruszewska, Warszawa (PL)

(21) Appl. No.: 14/404,793

(22) PCT Filed: May 29, 2012
(86) PCT No.: PCT/PL2012/000039

§ 371 Date: Dec. 1, 2014

(51) Int. Cl. C12R 1/225; A61K 35/747; A62C 2/06; A61L 29/08; A61L 29/16; A61L 29/00; C12N 1/20; A61K 45/06

(52) U.S. Cl. CPC C12R 1/225; C12N 1/20; A61K 35/747; A61K 45/06; A61L 29/085; A61L 29/16; A61L 29/005; A62C 2/06; A61L 2300/404; A61L 2300/41; A61L 2300/30; A61L 2300/428; A61L 2400/12

(57) ABSTRACT

The invention relates to a nanoproduct useful in prophylaxis and medicine, both human and veterinary as well as to a medical use of the same. The invention discloses Lactobacillus reuteri DAN080 strain with deposit number DSM 15693 for use in medicine. The various forms of preparations comprising the Lactobacillus reuteri DAN080 strain with deposit number DSM 15693 are disclosed for use in medicine as therapeutic and prophylactic agent, especially as an antimicrobial agent, in prophylaxis and treatment of medical condition conditions developing as a result of infections caused by bacteria, fungi, and other pathogens of the gastrointestinal tract, body surface, and other systems, such as urogenital system, respiratory system, in vertebrates or as a therapeutic and/or prophylactic agent for the treatment and prevention of development of gout (podagra) and/or for increasing the activity of lysozyme in an organism of vertebrate, especially human, other mammal or bird.

The present invention relates to a nanoproduct useful in prophylaxis and medicine, both human and veterinary as well as to a medical use of the same. A new microorganism isolated and identified by the present inventor—Danuta Kruszewska (DK), is a source of that product.

Applicant respectfully requests that a text file named MWA203SQ_ST25, created on Jul. 27, 2015, having a size 2,039 bytes, filed with the U.S. Patent and Trademark Office on Jul. 28, 2015, become a part of the present patent application via incorporation by reference.

Throughout the lifetime of a healthy human (and also of healthy animals), microorganisms are present in his/her organism. Among these microorganisms, bacteria prevail, although fungi and protozoa are also present. Various types of microorganisms colonize most intensively the mucosa of the gastrointestinal tract, body surface and other systems, e.g. urogenital or upper airways, and settle on mucosal surfaces. The mechanisms, according to which individual species/strains of a specific microbiota, as well as the microbiota as a whole perform their function in the maintenance of health, have not been fully recognized. Microbiota of the gastrointestinal tract, the skin, and other ecological niches inhabited by microbes differ with respect to the functions performed, composition and amount of microorganisms. Thus, the term ‘microbiota’ is to be understood as referring to a ‘herd’ of microorganisms inhabiting in a specific anatomical and physiological region of the body, which constitutes a microbiom of the microorganisms.

Conditions prevailing within the human gastro-intestinal tract will now be discussed in details to provide an illustration of various inter-relations and mechanisms that can be found also in other microbioms of living organisms.

Mutual interactions of the gastrointestinal tract microorganisms have the character of a symbiotic co-existence. Based on the example of the alimentary system, the microbiota of the system stimulate host immunity, not only on the level of a microorganism (live or dead), but also through its intra- and extra-cellular metabolites, counteract the anchoring of other microorganisms dangerous for health (mainly pathogens)—through competitiveness with respect to receptor and substrate, neutralize toxic intra- and extra-cellular metabolites of other microbes, regulate the development and physiological functions of the intestine (not only on the level of digestive functions), ferment indigestible, but energetically useful substrates, metabolize glycans and amino acids, and synthetize vitamins. The complex participation of the microbiota or disfunction of the same in development of various metabolic diseases, such as for example gout, have thus far been rarely taken into consideration.

Helicobacter pylori is a microorganism colonizing gastric and duodenal mucous membranes, responsible for a number of medical conditions, including gastritis and other related diseases, such as gastric and duodenal ulcer, stomach (gastric) cancer, duodenal cancer, intestinal disorders, disorders of the gastrointestinal tract (GIT) and diarrhoea which, in fact, afflict every representative of the human species living in the modern societies of Western countries. This is the main medical problem for ⅓ of the human population, which has growing medical, social and economic consequences. Over 10 million Americans suffer due to H. pylori-related gastritis; although applying more recent definitions of gastritis the number of afflicted patients is estimated to be 14-25 million, including those who have not yet experienced gastric ulcer or cancer, but already experience sufficiently positive pain symptoms. Despite the known role of H. pylori in the pathogenesis of gastritis, the development of peptic ulcer and adenocarcinoma (see: Hunt R H. The role of Helicobacter pylori in pathogenesis: the spectrum of clinical outcomes. Scand J Gastroenterol Supl. 1996; 220: 3-9), it is also suggested that certain strains of H. pylori may occur in nature as commensals (see: Misiewicz J. Is the only good Helicobacter a dead Helicobacter? Helicobacter 1997: 2S: S89-S91).

The National Institute of Health of the United States of America estimates that in 1990 the cost of treatment of the above-mentioned gastritis and other related diseases, e.g. gastric and duodenal ulcer, stomach (gastric) cancer, duodenal cancer, intestinal disorders, GIT disorders and diarrhoea, reached $20 billion. With the ageing of the population and an increasing prevalence of gastritis, in the Unites States alone it is anticipated that up-till 2020 just the medical costs associated with these medical conditions will be over $60 billion.

The current methods of treatment and prophylaxis of gastritis and other related diseases, such as the above-mentioned gastric and duodenal ulcer, gastric cancer, duodenal cancer, intestinal disorders, GIT disorders, and diarrhoea, the development of which is H. pylori related, are usually based on decreasing the HCl production in stomach, or killing generally all the bacteria with antibiotics. Table 1 shows the effectiveness of the to-date methods of treatment.

TABLE 1
Known and commonly applied therapeutic strategies for the treatment
of gastritis.

| Strategy | Antibiotics | H⁺(protons) inhibitors |
|---|---|---|
| Reduction of stomach | Elimination | Poor effect or lack of effect |
| colonization by H. pylori | | |
| Modulation of HCl | Lack of effect | Reduction of gastric secretion |
| secretion | | |

In the alimentary system there also occur beneficial lactic acid bacteria (LAB) which, as such, as well as their products, are considered a part of natural health ameliorating microbiota and as agents improving the health of the host and the intestine (see: Kullisaar T, Zilmer M, Mikelsaar M, Vihalemm T, Annuk H, Kairane C, et al. Two antioxidative lactobacili strains as promising probioticts. Int J Food Microbiol 2002; 72: 215-24).

LAB are generally recognized as safe (GRAS) bacteria. Despite the outstanding effect on the general health status, especially the effect on the GIT, LAB bacteria show an insufficient capability of GIT colonization after the period of breast suckling in mammals, and generally, for the whole life-time in birds. This is due, firstly, by the deficiency of suitable substrates for the growth of LAB after the period of breast suckling, and secondly—characteristic of bacterial wall which is incapable of producing fimbriae, etc., responsible for the bacterial adhesion to the GIT epithelium or to the GIT mucosa.

Antibiosis between LAB and H. pylori is rarely employed as a therapy, which is due to the low efficiency of such a treatment because LAB cannot easily colonize the gastric mucosa.

Many LAB are currently available in commerce, offered, as live cultures, food additives for humans and feed additives for animals, such as for example, Lactobacillus plantarum 299 v, and others. It is expected that the products of LAB growing in the alimentary system will be beneficial for the consumer of these bacteria. This is very often controversial because live LAB cultures are excellently eliminated and killed by antibacterial compounds of the host, which basically reduce the growth of bacteria in stomach and the upper part of intestine.

Various other medical disorders may also secondarily lead to the development of gastritis. Such medical disorders are presented in Table 2.

TABLE 2
Medical conditions and methods of treatment causing gastritis or other
related diseases, e.g. gastric and duodenal ulcer, gastric cancer,
duodenal cancer, intestinal disorders, GIT disorders and diarrhoea.

| Classical diseases | Drug, drug abuse, allergy |
|---|---|
| Type 1 diabetes | NSAID (non-steroid anti-inflammatory |
| | drugs) |
| Type 2 diabetes | Methotrexate |
| Inflammatory bowel disease | Antibiotics |
| Lymphoma | Excess of thyroid hormone |
| Primary billiary cirrhosis | Alcohol |
| Rheumatoid arthritis | Immobilization |
| Coeliac disease | Hypovitaminosis |

In mammals and chickens the proper functioning of stomach is a problem. Especially in the case of force-fed fast growing animals, with feeding a very non-physiological feed, the stomach does not develop correctly and many health disorders occur, the problems caused by various forms of gastritis, which is the cause of decreased efficiency of animal production. In order to prevent such conditions, which not only cause unnecessary suffering to animals, but also increase farmers' costs, a better understanding and control of physiological and pathological processes in the gastric mucosa—associated with the presence of H. pylori and other pathogens of the gastrointestinal tract, is necessary in the postnatal life of vertebrates, including mammals and birds.

In the light of the above indicated, known in the art inconveniences of the to-date therapies offering prevention and alleviation of gastric diseases and related conditions, as well as in view of high costs of treatment and correction of GIT function in conditions related to, for example, gastritis or gastric ulcer, there is a strong demand for development of a new product and improved methods and compositions for use in human and veterinary medicine in order to improve functioning of the stomach by elimination or regulation of the growth of H. pylori and other pathogens of the gastrointestinal tract.

Gout, also known as podagra [when it involves a big toe], is a metabolic disease usually characterized by recurrent attacks of acute inflammatory arthritis. The disorders are accompanied by severe pain experienced by a patient, caused by the presence of deposits in the form of crystals of uric acid sodium salt in the joint tissues and in many organs, among others, in the kidneys and urinary tract. Arthritis is manifested by redness, tenderness and swelling, and in half of the cases it affects the metatarsal-phalangeal joint of a big toe.

In the course of gout there may also occur tophi, which are deposits of urate salts in soft tissues, kidney stones (nephrolithiasis), nephropathies. An elevated level of uric acid (crystallization) in blood is accompanied by its deposition not only in joints, urinary tract, and surrounding tissues but also in tendons, ligaments, and cartilages.

Approximately 20% of gout patients have stones formed in the urinary system, kidney stones (gout nephropathy—in the parenchymal tissue of renal medulla and renal pyramids). Urolithiasis is caused by the precipitation of uric acid crystals in tubuli colligens, renal pelvises and ureters. Urate calculi constitute ca. 10% of all kidney stones.

Recently, urolithiasis has been widely developing in the countries with a higher standard of living. It is estimated that it affects 5.2-15% of males and 6% of females. An increase in morbidity is associated with the changes of life style, changes of nutritional patterns, and obesity becoming widespread. ‘Metabolic syndrome’ covers, apart from gout, such diseases as hypertensive disease, diseases associated with the lack of balance in metabolism of lipids, type 2 diabetes, cardiovascular diseases, which are accompanied by the formation of stones. Nutritional patters, i.e. the amount, composition and type of diet, affect food metabolism and absorption into the blood and lymph stream of nutritional substances. The result of these processes is the formation of stones, varied with respect to their chemical structure. Thus, together with a diet rich in oxalates, stones are formed based on calcium oxalate (75% of stones, struvite 10-20%, uric acid 5-6%, and cystine 1%).

Considering the fact that about 50% of patients with stone disease suffer due to recurrences, a metabolic and/or pharmacological prophylaxis of this disease is recommended (see: Porena M, Guiggi P, Micheli C. Prevention of stone disease. Urol Int. 2007; 79 Suppl 1:37-46).

The presence of crystals results from impaired excretion by the organism (reduced clearance) of the uric acid with urine, and more rarely, from an overproduction of uric acid. When the concentration of uric acid in serum (sUA) exceeds 6.8 mg/dL, extracellular fluids become saturated with this acid, which is defined as a state of hyperuricemia (see: Becker M A, Ruoff G E. What do I need to know about gout? J Fam Pract. 2010; 59(6 Suppl):S1-8).

The diagnostics of gout is based on the visualization of characteristic crystals present in joint fluid, and on the image of blood and urine.

It should be emphasized that gout affects approximately 1-2% of the population of highly developed countries, with an upward tendency observed in recent decades. Disorders of the uric acid metabolism are associated with innate genetic predispositions, disfunction/lack of enzyme activity, but also with such factors as an increase in the average life span, changes of dietary habits. Hyperuricemia is a disease of adults and women at post-menopausal age.

Throughout the lifetime of a healthy human (as well as other animals), microorganisms are present in his/her organism. Among these microorganisms, bacteria prevail, although fungi and protozoa are also present. Various types of microorganisms colonize most intensively the mucosa of the gastrointestinal tract, body surface and other systems, e.g. urogenital or upper airways, and settle on mucosal surfaces. The mechanisms, according to which individual species/strains of a specific microbiota, as well as the microbiota as a whole perform their function in the maintenance of health, have not been fully recognized. Microbiota of the gastrointestinal tract, the skin, and other ecological niches inhabited by microbes differ with respect to the functions performed, composition and amount of microorganisms. Thus, the term ‘microbiota’ is to be understood as referring to a ‘herd’ of microorganisms inhabiting in a specific anatomical and physiological region of the body, which constitutes a microbiom of the microorganisms.

Mutual interactions of the gastrointestinal tract microorganisms have the character of a symbiotic co-existence. Based on the example of the alimentary system, the microbiota of the system stimulate host immunity, not only on the level of a microorganism (live or dead), but also through its intra- and extra-cellular metabolites, counteract the anchoring of other microorganisms dangerous for health (mainly pathogens)—through competitiveness with respect to receptor and substrate, neutralize toxic intra- and extra-cellular metabolites of other microbes, regulate the development and physiological functions of the intestine (not only on the level of digestive functions), ferment indigestible, but energetically useful substrates, metabolize glycans and amino acids, and synthetize vitamins. Their complex participation or dysfunction in the case of the development of gout has not been considered to-date.

Lysozyme, a hydrolytic enzyme released by certain phagocytes, such as macrophages and multinuclear leukocytes, plays a significant role in the control of pathogenic microorganisms. Lysozyme is also produced by Paneth cells located in the lining of the intestines. Lysozyme is especially active against Gram-positive microorganisms. Phagocytic activity of cells involves degradation, with the participation of lysozyme, of the cellular walls of bacteria, more precisely—a cleavage of glycoside bonds in peptidoglycans.

Despite the possibilities of isolation and full identification of lysozyme, the therapeutic use of this compound is not possible due to its high activity. In the publication No. WO 89/11294 a possibility of the therapeutic use of the dimerized form of lysozyme is disclosed indicating its effectiveness in therapeutic use in human and veterinary medicine.

The treatment of gout to-date involves administration of anti-inflammatory agents reducing the intensity and duration of pain, in the form of non-steroid anti-inflammatory drugs (NSAIDs), steroids, or a classic agent—colchicine, alleviating the symptoms of the disease.

Colchicine has anti-inflammatory activity and decreases the level of uric acid concentration in organisms. The disadvantage of this drug is its toxic effect. Side effects take a form of diarhorrhea, strong pain in the abdominal integuments, and vomiting. In order to inhibit or considerably reduce attacks of gout, the administration of colchiciline in reduced doses is maintained, which reduced doses should not contribute to the development of side disorders; however, this does not provide a safe and effective therapy.

Colchicine metabolism involves participation of the cytochrome CYP 3A4 system. The CYP 3A4 inhibitors and glycoproteins P, such as antimicrobial antibiotics and antifungal drugs, i.e. claritromycin, erythromycin, ketokonazole, and cyclosporines, increase the concentration of colchicine. An interaction between colchicine and the antibiotics is then observed, and due to such interaction antimicrobial therapies with the above-mentioned agents are dangerous in the course of gout development (disruption of the striated muscles—rhabdomyolysis) (see: Finkelstein Y, Aks S E, Hutson J R, Juurlink D N, Nguyen P, Dubnov-Raz G, Pollak U, Koren G, Bentur Y. Colchicine poisoning: the dark side of an ancient drug. Clin Toxicol (Phila). 2010; 48(5):407-14).

Sometimes, alternatively non-steroid drugs having anti-inflammatory activity, such as phenylbutazole or indomethacin, are administered. They are, however, disadvantageous due to their side effects, such as bone marrow impairment and internal bleedings.

The majority of gout patients requires finally a long-term therapy decreasing a level of uric acid (urate-lowering therapy—ULT) achievable through a use of allopurinol and probenecid.

The treatment of attacks involves mainly administration of colchiciline, non-steroid anti-inflammatory drugs, and in exceptional cases—glucocortisteroids. In patients with an increased uric acid production allopurinol is administered.

Hyperuricemia is accompanied by the development of deposits and crystals. In addition, adsorption of pathogenic bacteria, their growth and states of recurrent inflammation of the urinary system frequently occur in the state of hyperuricemia.

For adsorption of the mineral deposits, uricosuric drugs, such as sulfinpirazon, and allopurinol (inhibiting the synthesis of uric acid) are administered. Due to such treatment, gout stones in the kidneys are dissolved, while those which have developed to a non-dissolvable size are removed by surgical treatment (e.g. laser).

Under the circumstances, there is a strong need for providing an agent which would support prophylactic actions, protecting against the development of this dangerous disease and increasing the effectiveness of the diet applied, or even eliminating the need for its application.

In human medicine, as well as in veterinary medicine and in animal husbandry, frequently development of undesired phenomena and medical conditions is being noticed, caused by microorganisms able to invade and colonize specific anatomically or physiologically defined body areas, including integument injured in result of burns and wounds, and also some medical equipment, instruments and even prostheses, which microorganisms are involved in pathogenesis of such undesired effects.

A representative example of medical equipment exposing a patient to such a risk is an urological catheter.

Catheters are widely used in human and veterinary medicine, as to enable drainage or administration of fluids or gases, and for insertion of endoscope, tubes and surgical instruments.

However, apart from undoubted benefits, undesirable effects are also associated with the use of catheters.

Despite the efforts towards maintaining the conditions of sterility and hygiene, the use of a catheter increases the risk of the entry of pathogenic agents from the external environment, as well as dissemination of the organism's own natural microbiota colonizing specific sites/niches in the organism, becoming pathogenic microbiota at the site of catheter placement. The occurrence of pathogens in the region of catheterization causes the development of infection (of various intensity).

In addition, the insertion of a catheter into a body vessel, duct or cavity is a procedure both painful and stressful for a patient. Moreover, as a result of a long-term contact of tissues with the catheter and agents flowing through the catheter there may develop inflammations, and frequently, also allergic reactions.

According to the intended use, catheters are produced of various plastics. Known catheters manufactured of medical polyvinyl chloride (PVC)—mainly of the Nelaton type, are used for a short-term catheterization and kept in a patient's body for a period up to 3 days. Such catheters constitute over 80% of the catheters used. Other polymers employed for production of catheters are polyurethanes (PU), silicon or natural latex and their derivatives. Mainly Folley catheters (with a fixation device) are produced of silicon, which Folley catheters are medical products for long-term use and may be kept in place for up to 3 months.

Unfortunately, high quality polyurethane and silicon catheters are considerably more expensive than those produced of PCV, and use of natural latex is while at present not recommended as it may cause allergic reactions.

Common problems connected with the necessity to use catheters in human and veterinary medicine are well illustrated by the phenomena accompanying the catheterization of patients with urological problems. Despite the achievements in medicine and health care, catheter-associated urinary tract infections (CAUTI) still remain among the most frequently occurring hospital infections. For example, in the USA each year, more than 1 million hospitalized patients need to be treated due to CAUTI.

A permanent indwelling urethral catheter is one of the causes of the occurrence of hospitals-acquired urinary tract infections (UTI). In many cases, hospital infections are caused by multidrug-resistant bacterial strains and require complex and expensive treatment with antibiotics.

Each year in the Unites States, catheters are inserted in more than 5 million patients in intensive-care units, and even more in nursing homes. In Poland, 200,000 urethral catheters are used annually. It is estimated that the risk of infection associated with short-term catheter use is 5-6% per one day of catheterization. Infections related with long-term catheterization lasting for more than 7 days are usually caused by a number of bacterial strains which form a biofilm on the catheter surface, and may lead to catheter clogging.

Biofilm is generated by various microbes, including ureolytic bacteria, non-pathogenic commensals, permanently and naturally colonizing the surface of the epithelium, pathogens, including microorganisms causing urogenital system infections (e.g. Proteus mirabilis). The common feature of ureolytic bacteria is their ability to make use of urea present in their environment (tissues)—mainly as a source of nitrogen necessary for survival, which use involves urease. Urease (including bacterial urease), hydrolyses urea to ammonia and carbon dioxide. Examples of nitrogen-assimilating bacteria by means of their own ureases are biofilm forming bacteria.

Ureolytic bacteria, even though they are not the main etiological factor in urinary tract infections in healthy organisms, are often associated with infections in patients with urinary tract disorders. Ureolytic bacteria are responsible for the forming of biofilm and mineralization of deposits on catheters and other medical instruments. Among the consequences of urinary tract infections caused by urease-producing microorganisms is nephrolithiasis, accompanied by supersaturation of urine with mineral salts: ammonium-magnesium phosphate (struvite), calcium phosphate, oxalates and urates. In physiological conditions, urea does not contain these salts in amounts indicating the formation of sand or stones.

The infected kidney stones formation is associated with the urinary tract infections caused by microorganisms of the following genera: Proteus, Ureaplasma, Klebsiella, Pseudomonas, Staphylococcus, Providencia, and Corynebacterium.

Another undesirable effects of these infections are pathological processes within the kidney parenchyma. Bacteremia is one of serious complications which may occur as a result of catheterization.

In general, morbidity due to urinary tract infections depends on the characteristics and status of a patient, the pathogenic microorganism and hospital environment. Usually, little can be done to reduce factors associated with the host (organism), because most of them are intrinsic either to a patient (host) or to bacteria.

Age, self-catheterization, and total or continuous incontinence by patients with neurogenic disorders are a few of the factors of hospital-acquired urinary tract infections. Such patients suffer as a result of infection associated with catheterization. In hospital conditions, important risk factors cover the type of catheter, duration of insertion, type of placement, and use of antibacterial or antiseptic substances. Microbes permanently colonizing the urinary tract represent the major source of microorganisms causing catheterization-related infections. A significant number of bacteria are found in urine samples, among them E coli strains prevail. The infection usually takes an asymptomatic course, while in 20% of cases it is symptomatic, irrespective of antibiotic treatment. The index of bleeding/blood traces ratio (connected with catheter insertion) is also high and occurs in one per five cases. Seventy five percent of patients catheterized for a period longer than one year develop UTI symptoms of varying intensity.

Patients of both sexes, especially the elderly undergoing long-lasting treatment with intermittent catheterization, usually complain also of physical and psychological complications related to the treatment.

Urethral catheters are usually manufactured from natural latex or synthetic polymers. Contemporary catheters available on the market differ with respect to shape, method of dilatation, and materials of which they have been manufactured. These characteristics cause differences in the protocols of use of individual catheters.

In the prior art, attempts have been undertaken to enhance the usability values of catheters to be applied in human and veterinary medicine by coating them with chemotherapeutics and impregnation with antiseptics or other agents (e.g. anticoagulants). The first remedy is the application of hydrogel on the catheter surface directly before catheterization in order to decrease its friction coefficient and reduce pain. It is mainly composed of polyvinyl pyrrolidone (PVP), usually in combination with iodide acting antiseptically. However, it requires additional operations and the gel applied is easily rinsed away, therefore the antiseptic action is limited in time. More advanced coatings are permanently attached to the catheter surface and allow decrease in the friction coefficient, not only during insertion, but also during catheter removal. A permanent hydrogel coating may also serve as an antimicrobial drug reservoir that slows down surface colonization. Nevertheless, the problem of infections associated with long lasting catheterization has not been fully resolved. In the case of long term placement of a catheter within the organism, the process of antibacterial agent release should be controlled and slow to maintain its bactericidal properties for the whole period of catheterization, which has not been accomplished in a sufficient degree.

There are many known methods of application of antibacterial coatings on natural or polymer tubes. Coating techniques vary as well. The hydrogel coating technique is advantageous due to high biocompatibility of the coating, low friction, decreased bacteria adhesion, and a possibility of incorporation of drug into the coating.

In EP 1917959 the use of alpha-ketoglutarate has been disclosed for production of an agent against the deposit and infectious stones formation in the urogenital system by ureolytic bacteria.

Despite the studies conducted and efforts undertaken to solve the above-mentioned problems, the whole clinical world awaits a solution which would enable furnishing patients with catheters, especially urethral catheters, for a long periods of time.

It is thus the main aim of the presented invention to provide a new line of therapeutic products and improved methods and compositions for use in human medicine in order to improve functioning of the stomach by elimination or regulation of the growth of H. pylori and other pathogens of the gastrointestinal tract.

Further aim of the invention is to provide a veterinary product and method for amelioration of the function of the stomach and remaining parts of the GIT in vertebrates, especially in mammals and birds, by means of elimination or regulation of the growth of H. pylori and other pathogens of the gastrointestinal tract.

It is also the aim of the invention to provide a new antibacterial and/or antimicrobial agent for use in prophylaxis and treatment of medical conditions developing as a result of infections induced by bacteria, fungi and other pathogens of the alimentary system, body surface, and other systems, such as the urogenital and respiratory systems in vertebrate, including human, other mammal and bird.

A still further aim of the present invention is to provide an agent useful in the prophylaxis and therapy of gout and other diseases of so-called metabolic syndrome, ensuring an improvement in salubrity of the populations of highly developed countries, and eliminating social and economic costs associated with the prevalence of these diseases.

Additional aim of the invention is to provide an agent enhancing activity of the immune system of the body, thus increasing immunity minimize diseases and side effects induced by pathogenic microorganisms, especially by viruses and bacteria.

The present invention is also aiming to provide specialist catheters which would be long-lasting, functional and simultaneously, appropriate for long-term keeping inside a patient's body and could reduce the risk of infection.

Another objective of the invention is the use of nanotechnology to provide natural antibacterial catheters, including urethral catheters.

Another aim of the invention is the use of nanotechnology to provide natural antimicrobials, which would provide the certainty of endogenous biodegradation, for body fluids and tissues, acting from the surface of catheters.

The above-mentioned and other goals were unexpectedly achieved due to the development of a new technical solution based on the isolation and identification of a new microorganism—Lactobacillus reuteri DAN080, deposited on Jun. 20, 2003—in accordance with a Budapest Treaty on the International Recognition of the Deposit of Microorganisms for the Purposes of Patent Procedure, in DSMZ collection—Deutsche Sammlung von Mikroorganismen and Zellkulturen GmbH, in Braunschweig, DE, access number: DSM 15693.

The invention relates to cultures of L. reuteri DAN080, the partially inactivated cultures of L. reuteri DAN080, liquid supernatants of L. reuteri DAN080 cultures, concentrated supernatants of L. reuteri DAN080 cultures, and dried supernatants of L. reuteri DAN080 cultures, for medical use, as therapeutic or prophylactic agent, especially as an antimicrobial agent for prophylaxis and treatment of medical conditions developed as an effect of infections caused by bacteria, fungi and other pathogens of the gastrointestinal tract, body surface, and other systems, such as the urogenital system, respiratory system etc., or else of various metabolic diseases in vertebrates.

The present therapeutic or prophylactic agent antimicrobial agent is selected from a group comprising the whole culture of L. reuteri DAN080, liquid supernatant, concentrated supernatant, and dried supernatant obtained from the culture of L. reuteri DAN080, and from mixed bacterial cultures comprising L. reuteri DAN080, and liquid supernatants, concentrated supernatants and dried supernatants obtained from cultures of prokaryotic and eukaryotic recombinants and whole cultures of prokaryotic and eukaryotic recombinants, in which and/or from which genes are utilized, which genes provide specific modulating, inhibitory, homeostatic activity against H. pylori and other bacteria, as well as proteins/oligopeptides/peptides of the molecular weight of approximately: 150 and/or 141 and/or 115 and/or 95 and/or 90 and/or 86 and/or 83 and/or 77 and/or 71 and/or 63 and/or 59 and/or 56 and/or 49 and/or 46 and/or 43 and/or 39 and/or 34 and/or 32 and/or 30 and/or 22 kD and lower, purified/isolated from liquid/condensed/dried supernatant obtained from L. reuteri DAN080 and purified/isolated from whole cultures of L. reuteri DAN080, and from other mixed bacterial cultures comprising L. reuteri DAN080, and purified or isolated from cultures of prokaryotic and eukaryotic recombinants, in which and/or from which genes are utilized, which genes provide specific modulating, inhibitory, homeostatic activity against H. pylori and other bacteria, or mixtures thereof for prophylaxis and treatment of medical conditions developed as an effect of infections caused by bacteria, fungi and other pathogens of the gastrointestinal tract, body surface, and other systems, such as the urogenital system, respiratory system in vertebrates, or else for treatment and prevention of the development of gout (podagra) and/or for increasing the activity of lysozyme in a body of vertebrate, especially human, other mammal and bird.

According to the invention the vertebrate is human individual.

The vertebrate is also a domestic animal, pet, animal involved in sport, broiler, layer hen, mouse, rat, guinea pig, rabbit and other laboratory animal, including primates, independently of their age.

According to the invention the microorganism is a pathogenic bacterium or fungi.

In particular, a pathogenic bacterium is H. pylori.

The invention relates also the use of the antimicrobial agent of the present invention for manufacturing a composition for modulation of function of stomach, intestine and GIT, or else for treatment and prevention of the development of gout (podagra) and/or for increasing the activity of lysozyme in a body of vertebrate in need of such a treatment, including humans, other mammals and birds, which composition comprise the agent in an effective amount, that provides for obtaining the desired preventive or therapeutic effect.

According to the invention the composition is in particular to kill, inhibit, regulate, and prevent the growth of H. pylori and other microorganisms, or else for treatment and prevention of the development of gout (podagra) and/or for increasing the activity of lysozyme in a body of vertebrate, especially human, other mammal and bird, and to be administered in an effective amount and at a sufficient rate, necessary for reaching the desired preventive or therapeutic result.

Preferably, the composition is intended for treatment, alleviation or prophylaxis of GIT disorders, gastritis, gastric ulcer, duodenal ulcer, gastric cancer, and duodenal cancer, or else for treatment and prevention of the development of gout (podagra) and/or for increasing the activity of lysozyme in a body of an individual—a vertebrate, including humans, other mammals and birds, in need thereof.

In particular, the composition is a pharmaceutical composition comprising optionally, other biologically active substances, such as vitamins, especially vitamins D and E, especially in a nanoform, salts of lactic acid and other acids comprised in the L. reuteri DAN080 metabolites, in preventive or therapeutic doses, and pharmaceutically acceptable carriers and/or additions.

Preferably, the pharmaceutical composition is in a solid form and is divided into single doses comprising therapeutically effective amount of the present therapeutic or prophylactic agent, in the amount of from 0.001 to 0.2 g/kg of body weight per day. The composition has in particular a form of a tablet or capsule.

Alternatively, the pharmaceutical composition of the present invention is in a liquid form and is divided into single doses comprising a therapeutically effective amount of the present therapeutic or prophylactic agent, in the amount of from 0.001 to 0.2 g/kg of body weight per day, especially in an ampoule. Such composition has a liquid form for use as aerosols, cataplasm or moist compress.

Especially preferably, in accordance with the invention a product of fermentation of Lactobacillus reuteri DAN080 is used in form of cultures, at least partially inactivated cultures and the supernatants of these cultures—respectively processed, for the modulation of the function of stomach, intestine and GIT, or else for treatment and prevention of the development of gout (podagra) and/or for increasing the activity of lysozyme in a body of vertebrate, especially human, other mammal and bird—i.e. in vertebrates in need for such treatment, including humans, other mammals and birds.

Although the present invention is discussed with reference to the exemplary influence exerted on H. pylori—as a pathogenic agent present in the human gastrointestinal tract, when administered per os, the demonstrated biological activity of live cultures of the new bacteria L. reuteri DAN080 and the above mentioned derived forms, allows those skilled in the art to use the solution according to the invention in the prophylaxis and treatment of pathological conditions developed under the influence of other pathogens in other systems of the human body and those of other mammals and birds, and with different routes of administration.

Preferably, in another aspect of the invention, the composition is a dietary supplement, food or beverage. The dietary supplement, food or beverage is in a solid form and/or in form of beverage. Preferable amount of the therapeutic and prophylactic agent of the present invention is from 0.001 to 0.2 g/kg of body weight per day. Such a dietary supplement, food or beverage, and optionally contains other biologically active substances and vitamins D and E, especially in the form of nanoparticles, in preventive doses.

The solution according to the invention allows the restoration of the normal metabolism of proteins and purine compounds—of utmost importance in gout prophylaxis, and has a stimulatory effect on the immune system, especially by increasing lysozyme activity and enhancing its antimicrobial and antiviral activity.

As it has been mentioned above, the present invention is also directed to special catheters. At present, it has been unexpectedly find out that the above-mentioned and other goals may be achieved by a solution according to the invention, based on coating the surface of a catheter with nanocoatings of various substances, primarily with the use in the composition of at least one nanocoating of a component originating from a new strain of lactic acid bacteria, decreasing stress associated with insertion and removal of catheters, and inducing a patient's immune response, thus reducing the risk of viral and bacterial infections.

Surprisingly, it was also observed that incorporation of specific vitamins in nanoform and alpha-ketoglutarate into nanocoatings of a catheter according to the present invention provides a synergistic effect with respect to the prevention of infections and inflammations of the tissues remaining in a long-term contact with the catheter.

Catheter for insertion into body vessels, ducts and/or cavities, for use in prophylaxis, diagnostics and medicine, both human and veterinary, made of plastic and coated with a protective lubricant layer, in accordance with the invention has an outer nanocoating of biocompatible polymer capable of forming gel with water, permanently attached to said plastic either directly or through a nanocoating of polymer chemically bonded to the catheter material and having antibacterial properties, wherein at least one of the nanocoatings comprises an addition of extracellular metabolites secreted by Lactobacillus reuteri DAN080, said metabolites having antimicrobial and anti-inflammatory activity, as well as an optional addition of vitamin D and E in form of nanoparticles.

According to the invention the biocompatible polymer is polyvinyl pyrrolidone and thickness of the nanocoating made of this polymer is about 50,000 C—C bonds (10 nm).

Preferably, the polymer having antibacterial properties is a salt of chitosan and small organic acid, preferably alpha-ketoglutaric acid.

According to the invention, in nanocoating of biocompatible polymer and/or in nanocoating of polymer having antibacterial properties there are dispersed additional active agents selected from the group comprising chitosan alpha-ketoglutarate, chitosan citrate, chitosan lactate with antimicrobial and anti-inflammatory activity, small di-carboxylic acid, silver nanoparticles, vitamin D and E in the form of nanopowder coated with a protective coating and combinations thereof.

The invention also covers a kit for catheterization, comprising a catheter and a vial with water for injection (sterile) and stress-reducing agent to be administered orally in the form of live or thermally inactivated cultures of Lactobacillus reuteri DAN080 at a dose of 10⁶ cells, for daily administration for the period of catheterization, preferably for oral administration 8 hours prior to catheterization, or for administration into the body cavity 15 minutes before catheterization.

In the kit according to the invention, the water container is preferably fixed at the catheter tip, and the container has a partition separating water from the catheter, the partition being destroyed by rotation of the container against the catheter while protruding the catheter from the packaging.

The catheter according to the invention satisfied all the above-mentioned requirements and is user-friendly, acceptable for the catheterized patients, the medical staff and medical opponents. According to the invention nanocoatings were developed for on catheters made of PVC and of silicon. The hydrogel nanolayer is permanently attached to the polymer surface, and not only decreases the friction coefficient, thus reducing pain experienced by the patient catheterized, but also contains an additive being an agent which in contact with the patient's body tissues reduces patient's stress associated with catheter insertion and removal. Simultaneously, the same additive induces an increase of lysozyme level in tissues remaining in contact with the catheter, thus decreasing the risk of viral, bacterial infection, due to a very wide spectrum of lysozyme activity. The presence of a new coating on the catheter surface reduces the formation of biofilm on the catheter surface. This nanocoating contains also other active substances released slowly in a controlled manner.

Surface nanoengineering applied in accordance with the present invention allows providing a coating which releases a medicament on demand. One of the signals triggering release of the drug may be, for example, a change of pH of the environment caused by bacterial growth. This targeted drug release is much more effective and shows less side effects. In addition, by the proper selection of the composition of coating layers dedicated catheters are obtained, having varied destination, adjusted to the patient's status. The use of a coating made of PVP/chitosan salt with a chemically bonded drug (e.g. small dicarboxylic acid) ensures achievement of the goals assumed.

The present catheter ensures the expected progress in medical care due to the possibility of making use of nanotechnology for delivery of natural antimicrobial agents, acting on the surface of the catheter according to the invention. The catheter being coated with nanocoating is more convenient in use and safer for a patient. The coating reduces pain connected with insertion of the catheter and significantly decreases the probability of infection. Moreover, all the active substances used do not induce any undesirable side effects in patient.

Due to the present invention based on the use of nanotechnology, the role of protective coatings on catheters is maximized thanks to enhancement of antimicrobial properties of nanocoatings and targeted release on demand of an active substance. It is most important that the catheters according to the invention will save pain for catheterized patients and decrease the number of infections, while in view of character of the compound employed eliminate the development of drug-resistance in microorganisms induced by classic antibiotics.

Finally, the present new therapeutic and prophylactic agent can also be used in the form of a dressing or hygienic materials for use in a personal hygiene, saturated with the agent exerting its antimicrobial activity.

The present new therapeutic and prophylactic agent is particularly advantageously used in form of a coating—preferably having a form of a nanolayer, on plastic protectors and fire blankets intended for rescue units, including fire rescue units, specifically for patients with severe burns and road traffic accident victims with extensive body injuries.

The present new therapeutic and prophylactic agent can be used for superficial (topical) applications on the body for its remarkable antimicrobial activity, also together with different absorbents including liquid absorbent material. Non-limiting examples of corresponding products include diapers, tampons, bandages, bandaids, sanitary pads, sanitary napkins with wings, panty liners, cosmetic pads, wraps for animals for night and day use. This group of articles can be composed of fibers, ultrathin (silk-thin soft) cotton surface layers. The present antimicrobial agent incorporated in diapers can help in the prevention of hip rash, tender skin care. Such diapers in various sizes, with adjustable buckle can be used by children and adults.

The fire blankets can be manufactured according to the new European standards for fire blankets. They are designed with extra flexibility, using specially selected materials coated or containing the present new therapeutic and prophylactic agent and adjusted to smother flaming objects or clothing. They can be fully asbestos free and will not fray. The fire blankets can be packed into quick release flexible wallets but can also be packed into containers, depending on the final users' requirements. They can take different size up to 180 cm². It is foreseen that each square e\centimeter of surface can release in use an effective amount of the present new therapeutic and prophylactic agent.

The benefit of this particular aspect of the present invention is such that when a potentially tragic in-door fire disaster takes place, it provides an immediate therapeutic treatment to burn victims that might require primary reconstructive burn surgery. Burn patients often have burns of differing sizes (smaller vs larger percentage of total body surface area) and degrees of severity, with some requiring escharotomies, fasciotomies, primary excisions, skin grafts, amputations, local flaps, free flap coverage, thoracic surgery, etc. with a long stay in hospital for more than 150 days. Severe infections can also occur if not immediately prevented.

Further goals and advantages resulting from the present invention will be discussed in details below in the detailed description of the invention, with the reference to the drawings, wherein:

FIG. 1—presents in graphic form the results of plate diffusion assay (agar medium GAB-CAMP with the strain H. pylori 17874): 1—non-active supernatant obtained from the culture of L. reuteri; 2—non-active broth MRS; 3 and 4—inhibition zones caused by the activity of the active supernatant obtained from the culture of L. reuteri DAN080;

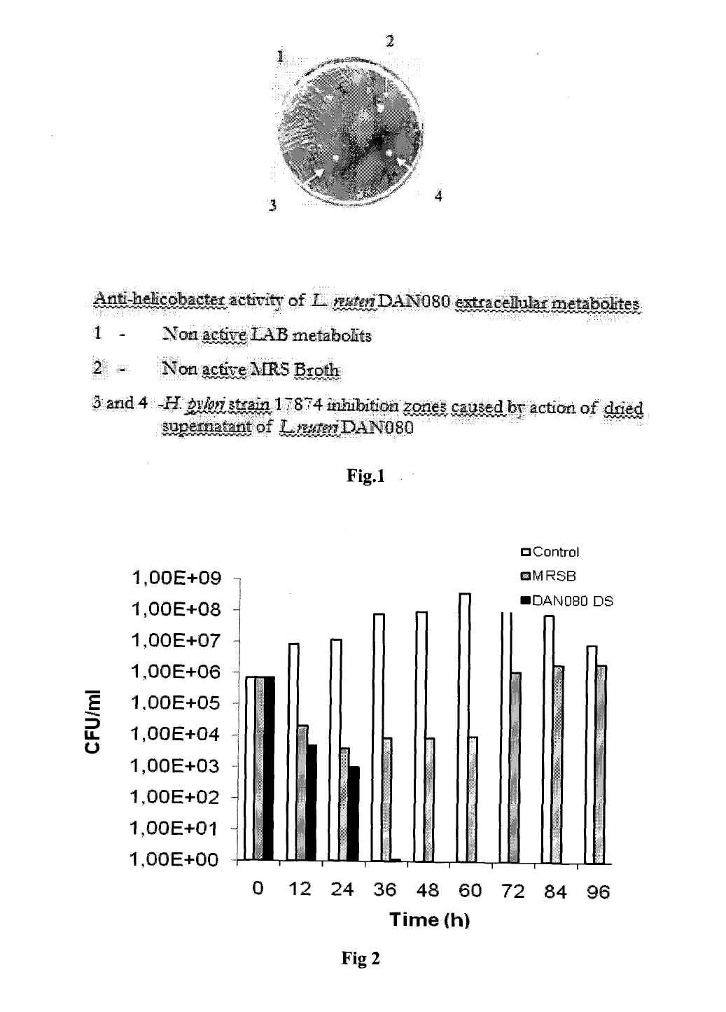

FIG. 2—illustrates the effect of supernatant from the culture of L. reuteri DAN080 on the growth of H. pylori in a liquid medium—BHI broth;

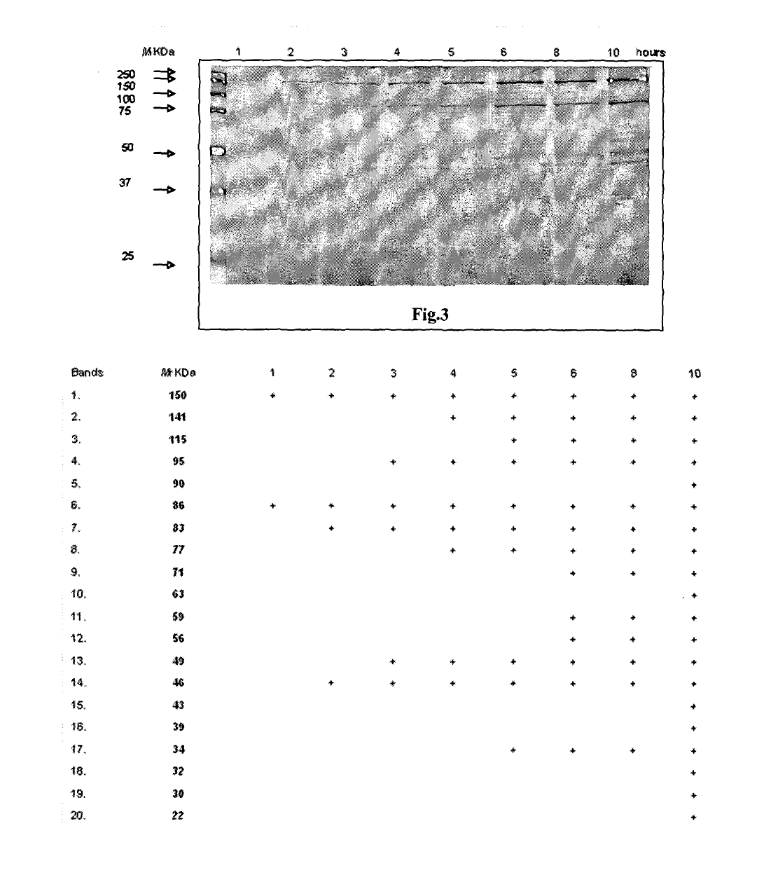

FIG. 3—presents SDS-PAGE electrophoresis of supernatants from L. reuteri DAN080 obtained, respectively, after 1, 2, 3, 4, 5, 6, 8 and 10 hours of growth in MRS broth. Numbers 1-20 designate the identified proteins released into the medium by L. reuteri;

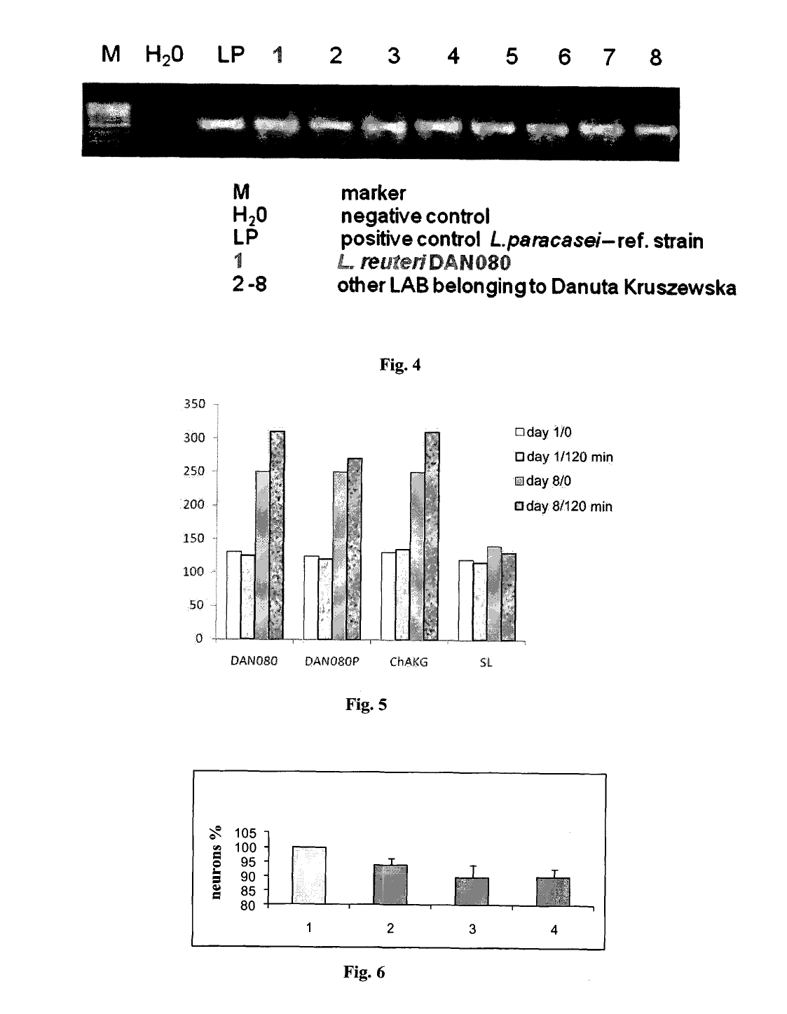

FIG. 4—illustrates the identification of L. reuteri DAN080 by Denaturating Gradient Gel Electrophoresis L. reuteri DAN080 PCR product amplified with primers LacF and LacR;

FIG. 5—shows the relationship between the activity of lysozyme (U/L) in rat blood, and the presence of the bacteria L. reuteri DAN080 in their gastrointestinal tracts, following intragastric administration of DAN080—10⁶ cells Lactobacillus reuteri DAN080; DAN080P—thermally killed 10⁶ cells Lactobacillus reuteri DAN080; ChAKG—chitosan alpha-ketoglutarate; SF—saline;

FIG. 6—presents the relationship between the activity of neurons isolated from the Enteric Nervous System and extracellular metabolites of L. reuteri DAN080;

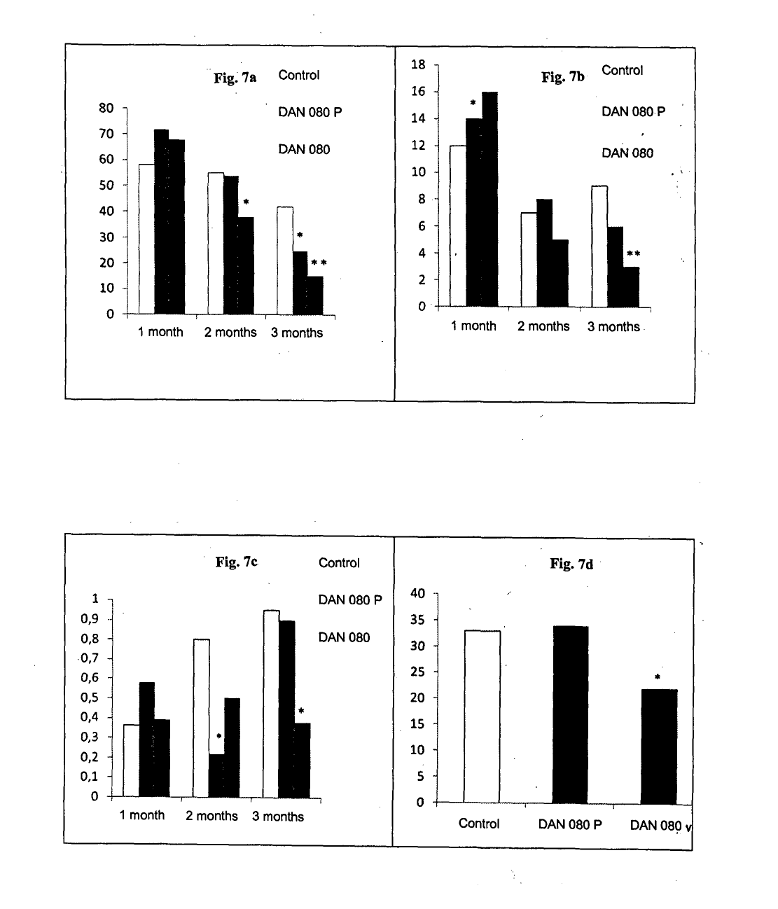

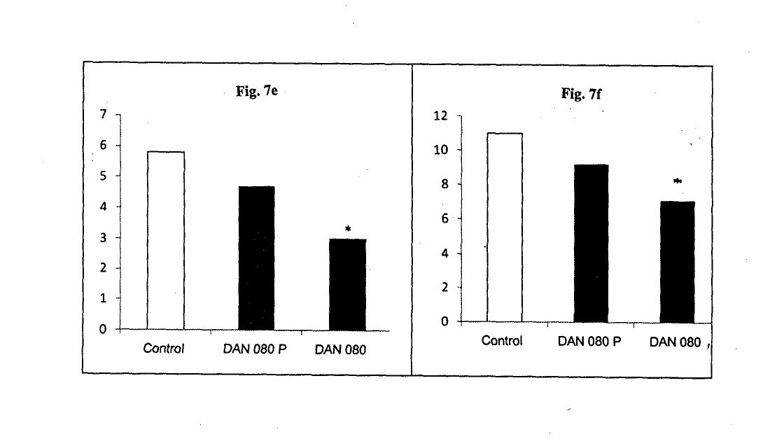

FIG. 7a-7f—illustrate the results of open field tests performed on rats treated with L. reuteri DAN080, inactivated L. reuteri DAN080, chitosan alpha-ketoglutarate, and of open field behavioral tests performed on rats on rats treated with L. reuteri DAN080, inactivated L. reuteri DAN080, and chitosan alpha-ketoglutarate and saline.

### DETAILED DESCRIPTION OF THE INVENTION

Bacteria L. reuteri DAN080 were isolated from the gastrointestinal tract of a healthy laboratory animal. The bacteria were isolated as a single colony on a solid medium with blood agar. This medium was incubated with the scrapings from the gastrointestinal tract of a healthy mouse at the temperature of 37° C. for 24 hours. The colony isolated was proliferated in the broth MRSB (Oxoid), on a standard medium for lactic acid bacteria (LAB). The pH of the medium prior to sterilization was pH 6.8. Sterilization was performed within 15 min., at temp. of 121° C., the pH of the medium after sterilization: pH 6.2. The thermal conditions of bacteria incubation remained within the range 35±3° C. The full growth in the liquid culture was 16 hours. The bacteria can be stored, with warranty of survival at the temperature of −20° C. L. reuteri DAN080, survive at room temperature+20 to +22° C. for at least 30 days maintaining the capability of inhibiting the growth of other microorganisms.

In order to obtain an enhanced antimicrobial activity the bacteria L. reuteri DAN080 are grown on media comprising AKG.

Composition of the medium: 0.5% meat extract; 0.5% yeast extract; 1% peptone; 0.3% NH₄Cl; 0.4% K₂HPO₄; 0.4% KH₂PO₄; 0.01% MgSO₄×7 H₂O; 0.005% MnSO₄×4 H₂O; 0.1% Tween 80; 0.05 L-cysteine HCl; 0.0002 of each of the following vitamins: B1, B2, B6, B5, B12, B9. After sterilization in the conditions as presented above, 23 mmol/1 maltose and, respectively, starch or glucose, and 10 mmol/1 AKG were added to the medium. The medium pH was: pH 6.2. The bacteria were incubated at 37° C. for 16 hours. An enhanced bacterial growth occurred in the presence of AKG. In the medium, from 4 to 6 mmol/1 of acetates and lactates were found, while in the medium non-enriched with AKG—from 1 to 2 mmol/1.

Lactobacillus reuteri DAN080 was identified in accordance with biochemical activity, where the capability of fermenting carbohydrates was assessed by the test—Api 50 CH and CHL medium, bioMerieux SA, Marcy lÉtoile, France.

Taxon: L. reuteri

phenotypic characteristics according to the API system

### CAT: 1053 1121 0000 000 000 0000000

### API RID32s: 515 151 511 111 315 111 111 511 111 111 11 1

### API 50CHL: 1111533111 5511111111 1411111155 5511151111 1111111311

### Api ID32AN: 155 515 111 114 111 513 351 351111 312

Genotypic characteristics [SEQ. ID NO: 1, 2, 3]: based on DNA analysis and comparison with 16S gene sequence of rhibosomal RNA L. reuteri (see: GenBank: EF187261.2; Byun R, Nadkarni M A, Chhour K L, Martin F E, Jacques N A, Hunter N. Quantitative analysis of diverse Lactobacillus species present in advanced dental caries. J Clin Microbiol. 2004; 42(7):3128-36; Fredricks D N, Relman D A. Improved amplification of microbial DNA from blood cultures by removal of the PCR inhibitor sodium polyanetholesulfonate. J Clin Microbiol. 1998; 36(10):2810-6). The following primers were useful for sequencing [SEQ. ID NO: 2, 3]—primer 1: TGGAAACAGA TGCTAATACC GC (22 bp) [SEQ. ID NO: 2], primer 2: ATTAGATACC CTGGTAGTCC (20 bp) [SEQ. ID NO: 3]

[SEQ. ID NO: 1]
tggaaacaga tgctaatacc gcataacaac aaaagccaca
tggcttttgt ttgaaagatg gctttagcta tcactctggg
atggacctgc ggtgcattag ctagttggta aggtaacggc
ttaccaaggc gatgatgcat agccgagttg agagactgat
cggccacaat ggaactgaga cacggtccat actcctacgg
gaggcagcag tagggaatct tccacaatgg gcgcaagcct
gatggagcaa caccgcgtga gtgaagaagg gtttcggctc
gtaaagctct gttgttggag aagaacgtgc gtgagagtaa
ctgttcacgc agtgacggta tccaaccaga aagtcacggc
taactacgtg ccagcagccg cggtaatacg taggtggcaa
gcgttatccg gatttattgg gcgtaaagcg agcgcaggcg
gttgcttagg tctgatgtga aagccttcgg cttaaccgaa
gaagtgcatc ggaaaccggg ccacttgagt gcagaagagg
acagtggaac tccatgtgta gcggtggaat gcgtagatat
atggaagaac accagtggcg aaggcggctg tctggtctgc
aactgacgct gaggctcgaa agcatgggta gcgaacagga
ttagataccc tggtagtcc (659 bp)

After a specified time of L. reuteri DAN080 growth, the culture is centrifuged and the liquid supernatant, concentrated supernatant, and dried or lyophilized supernatant is the product of a specific capability and activity for regulating the growth of H. pylori and other bacteria in vitro and in vivo. After electrophoretic separation performed from the liquid supernatant, the concentrated supernatant and dried supernatant collected at a predetermined time, specific proteins of a molecular weight within the range 150-22 kD or less were visualized, which proteins are responsible for homeostasis and regulation of the growth of H. pylori. These bands are weak, and do not occur in electrophoregraphs obtained from liquid supernatant, concentrated supernatant and dried supernatant harvested at a time different than the above-specified time for L. reuteri DAN080 culture, and the liquid supernatant, concentrated supernatant and dried supernatant, show one of the proper effects—homeostatic and growth regulating, on H. pylori and other bacteria in vitro, as well as in vivo.

For the genetic identification of L. reuteri, the total genomic DNA was isolated from bacteria cultured overnight, with the use of the kits DNaesy™, Qiagen GmbH, Hilden, Germany.

Amplification of the 340 bp fragment of 16S rDNA was performed with semi-nested PCR (first run: 94° C. for 30 s., 61° C. for 60 s., 68° C. for 60 s., 35 cycles; second run: 94° C. for 30 s., 58° C. for 60 s., 68° C. for 60 s., 40 cycles) using primers [SEQ. ID NO: 4, 5, 6] (see: Walter, J., Hertel, Ch., Tannock G W., Lis C M., Munro K., Hammes W. P. (2001) Detection of Lactobacillus, Pediococcus, Leuconostoc i Weisella species in human faeces by using group specific PCR primers and denaturing gradient gel electrophoresis. Appl. Environ. Microb. 67, 2578-2585); forward primer 3: AGCAGTAGGG AATCTTCCA (19 bp) [SEQ. ID NO: 4]; reverse primer 4: ATTYCACCGC TACACATG (18 bp) [SEQ. ID NO: 5]; forward primer 5: ACAATGGACG AAAGTCTGAG TG (22 bp) [SEQ. ID NO: 6].

### DEFINITIONS

The terms used in the presented description should be understood in their common basic meaning, unless defined otherwise below.

As used herein, the term ‘killing, inhibiting, regulating, preventing growth of H. pylori and other bacteria product’, is intended to mean the pharmacological, chemical, mechanical and physiological characteristics of L. reuteri DAN080 cultures, partially inactivated L. reuteri DAN080 cultures, liquid supernatants of L. reuteri DAN080 cultures, condensed supernatants of L. reuteri DAN080 cultures and dried supernatants of L. reuteri DAN080 cultures, as measured by means of certain parameters applied in accordance with the invention. Such parameters are known to those skilled in the art, and are further defined in the presented description.

As used herein, the term ‘improvement in gastritis and other related diseases, e.g. gastric and duodenal ulcer, gastric and duodenal cancer, intestinal disorders, GIT disorders, and diarrhoea by the elimination or stabilization of H. pylori growth’ is intended to mean the chemical and physiological characteristics of the stomach, intestine, and GIT, as measured by means of certain parameters applied in accordance with the invention. Such parameters are known to those skilled in the art and are further defined in the presented description.

The term ‘improvement in gastritis and other related diseases, e.g. gastric and duodenal ulcer, gastric and duodenal cancer, intestinal disorders, GIT disorders, and diarrhoea by the elimination or stabilization of H. pylori growth’, is intended to additionally mean, in the present description, changes in mechanical, chemical and physiological characteristics of stomach, intestine and GIT functioning, thus defining the quality of the stomach, intestine and GIT as compared to those of vertebrates, including mammals and birds, with respect to which no prophylaxis and/or treatment is applied, or which are not administered in accordance with the present invention, with any culture of L. reuteri DAN080, partially inactivated L. reuteri DAN080 culture, liquid supernatant of L. reuteri DAN080 culture, concentrated supernatant of L. reuteri DAN080 culture and dried supernatant of L. reuteri DAN080 culture. The changes are considered as an improvement if such changes are positive for vertebrates, including mammals and birds.

The term ‘improvement in gastritis and other related diseases, e.g. gastric and duodenal ulcer, gastric and duodenal cancer, intestinal disorders, GIT disorders, and diarrhoea by the elimination or stabilization of H. pylori growth’, as intended in the present description may also mean a change, modification or other effect on the current mechanical, chemical and physiological characteristics of the stomach, intestine, GIT and colonization of the same by H. pylori.

According to the meaning defined in the present description and claims, the term ‘pharmaceutical composition’ means therapeutically and/or preventively effective composition of the invention comprising L. reuteri DAN080 cultures, partially inactivated L. reuteri DAN080 cultures of, liquid supernatants of L. reuteri DAN080 cultures, concentrated supernatants of L. reuteri DAN080 culture and dried supernatants of L. reuteri DAN080 cultures.

The term ‘therapeutically effective amount’ or ‘effective amount’ or ‘therapeutically effective’ applied in the presented description and claims refers to the amount of antimicrobial agent of the invention, that provides the therapeutic and/or preventive result when used in a specific condition and a specific administration regime. The term means a predetermined amount of the active material calculated so as to produce the desired therapeutic and/or preventive effect. The above-mentioned active material may be combined with an appropriate additive, for example, other microorganisms or diluent, or carrier or administration vehicle. In addition, the term is intended to mean an amount sufficient to reduce, and most preferably to prevent a clinically significant deficiency in vertebrates in need of such treatment, the vertebrates including mammals and birds. The establishment of a therapeutically effective amount remains within the scope of skills of a person skilled in the art and depends on the activity of the product according to the invention, place of activity, and innate sensitivity of vertebrates in need of the treatment, such vertebrates including mammals and birds. Alternatively, a therapeutically effective amount is sufficient to cause an improvement in the host's clinically significant condition.

As used herein, ‘treatment’ means treatment in order to cure, which may be a complete or partial recovery from the condition or states related to gastritis, gastric and duodenal ulcer, gastric and duodenal cancer, intestinal disorders, GIT disorders, and diarrhoea.

As used herein, the term ‘alleviation’ means the reduction, i.e. less severe or milder condition or states related to gastritis, gastric and duodenal ulcer, gastric and duodenal cancer, intestinal disorders, GIT disorders, and diarrhoea.

As used herein, the term ‘prevention’ or ‘prophylaxis’ means a complete or partial inhibition of the development or outbreak of the defined state or states related to gastritis, gastric and duodenal ulcer, gastric and duodenal cancer, intestinal disorders, GIT disorders and diarrhoea. Determination of a preventively effective amount remains within the scope of skills of a skilled artisan, and depends on the activity of the product, place of activity and innate sensitivity of an individual vertebrate in need of such treatment, the vertebrate including a mammal and bird. Alternatively, a preventively effective amount is sufficient to protect the host against the conditions related to gastritis, gastric and duodenal ulcer, gastric and duodenal cancer, intestinal disorders, GIT disorders, and diarrhoea.

With respect to the other a.m. aspects of the present invention it is essential to properly understand the following terms:

“Nanocoating” means known nanogel (polyvinyl pyrrolidone—PVP or other), in which polymers used are chemically bonded to chitosan salts of antimicrobial activity (citric or lactic or alpha-ketoglutaric acid salts, or mixtures thereof in various proportions and amounts).

Chitosan salts of antimicrobial activity (citric or lactic or alpha-ketoglutaric acid salts, or mixtures thereof in various proportions and amounts) may also form a coating which may be coated with a known nanogel in a non-modified form or modified with the above-mentioned chitosan salts of antimicrobial activity.

The subsequent coating may form vitamin D alternatively with chitosan salts of antimicrobial activity (citric or lactic or alpha-ketoglutaric acid salts, or mixtures thereof in various proportions and amounts), and a final coating of hydrogel in non-modified form or modified with chitosan salts of antimicrobial activity (citric or lactic or alpha-ketoglutaric acid salts, or mixtures thereof in various proportions and amounts).

Also possible is separate stratification of the catheter with extracellular metabolites of L. reuteri DAN080 as deposited under the number: DSM 15693 and with silver or modification therewith the nanogel which is coating the catheter on a side facing the site of catheter activity.

Development of the new therapeutic and prophylactic agent: In its first aspect, the presented invention concerns the development of a new therapeutic and prophylactic agent for the treatment, alleviation or prevention of the growth of H. pylori and other bacteria in various medical conditions. The conditions which come to mind with respect to the efficiency of the product in the treatment, alleviation or prevention of the growth of H. pylori and other bacteria in various medical conditions are, although are not limited to, gastritis and other related diseases, e.g. gastric and duodenal ulcer, gastric and duodenal cancer, intestinal disorders, GIT disorders, and diarrhoea.

According to the invention obtaining an improvement in the health status and in functioning of stomach and intestine, and GIT in vertebrates, including mammals and birds, is due to administration to vertebrates, including mammals and birds, a sufficient amount and optionally, at a sufficient rate capable of inducing the desired effect, cultures of L. reuteri DAN080, partially inactivated cultures of L. reuteri DAN080, liquid supernatants of L. reuteri DAN080 cultures, concentrated supernatants of L. reuteri DAN080 cultures and dried supernatants of L. reuteri DAN080 cultures.

The changes improving the health status and functioning of stomach, intestine, and GIT in vertebrates subjected to the treatment are compared with the health status and functioning of stomach, intestine and GIT in vertebrates which are not recipients of the antimicrobial agent of the invention. The changes are considered as an improvement if they are beneficial for the vertebrates in need of such treatment, including mammals and birds.

According to the present invention, L. reuteri DAN080 cultures, partially inactivated L. reuteri DAN080 cultures, liquid supernatants of L. reuteri DAN080 cultures, concentrated supernatants of L. reuteri DAN080 cultures and dried supernatants of L. reuteri DAN080 cultures as used as the new therapeutic and prophylactic agent.

The present new therapeutic and prophylactic agent according to the invention is used in treatment of vertebrates including mammals and birds—for instance, humans, domestic animals, pets, animals involved in sports, broilers, layer hens, mice, rats, guinea pigs, rabbits and other laboratory animals, including primates, independently of their age.

Administration of Liquid Supernatant, Condensed Supernatant and Dried Supernatant Obtained from the L. reuteri DAN080 Culture:

Administration may be accomplished via various routes selected according to the type of vertebrate to be treated, the condition of the vertebrate in need of the treatment by the above described new therapeutic and prophylactic agent and the specific indication for treatment.

In accordance with one solution, the agent is administered in the form of food or feed additive, such as dietary supplement and/or component in solid form and/or in the form of a beverage. Further solutions may be in the form of suspensions or solutions, such as the beverages further described below. Suitable forms may also be aerosols, globules, suppositories, capsules or tablets, chewable or soluble, e.g. effervescent tablets, as well as powders and other dry forms known to those skilled in the art, such as granules, for example microgranules.

The administration may be parenteral, rectal, intravaginal, inhalatory and oral, in the form of additives to feed or food, as disclosed herein above. Vehicles for parenteral administration include sodium chloride solution, Ringer's solution with dextrose, dextrose and sodium chloride solution, Ringer's solution with lactates or plant oils.

Feed and feed additive may be also emulsified. The therapeutically active component may subsequently be mixed with pharmaceutically acceptable excipients compatible with the active component. Suitable excipients are, for example, water, saline, dextrose, glycerol, ethanol, or the like, and combinations thereof. In addition, if desired, the composition may contain trace amounts of auxiliary substances, such as lubricating or emulsifying agents, pH modulating agents, buffering agents, which enhance the effectiveness of the active component.

Various forms of feeds or feed additives may be provided, such as solid, liquid, lyophilized, or dried otherwise. They may include diluents as for example various buffers (e.g., Tris-HCl, acetate, phosphate buffers) having various pH ranges and ionic strength, additives, such as albumin, gelatin, detergents (e.g. Tween 20, Tween 80, Pluronic F68, bile acid salts), solubilizing agents (e.g. glycerol, polyethyleneglycerol), antioxidants (e.g. ascorbic acid, sodium metabisulfite), preservatives (e.g. thimerosal, benzyl alcohol, parabens), bulking substances or tonicity modifiers (e.g. lactose, mannitol), polymers, such as polyethylene glycol, polymers forming complexes with metal ions, polylactic acid, polyglycolic acid, hydrogels, etc., or liposomes, nanocapsules, microemulsions, micelles, unilamellar or multilamellar vesicles, erythrocyte ghosts, spheroplasts or chitin derivatives.

Beverages:

In one solution, the feed or feed additive is administered in the form of a beverage, or its dry formulation, by any method disclosed.

The beverage contains an effective amount of the product in the form of L. reuteri DAN080 cultures, partially inactivated L. reuteri DAN080 cultures, liquid supernatants of L. reuteri DAN080 cultures, concentrated supernatants of L. reuteri DAN080 cultures, and dried supernatants of L. reuteri DAN080 cultures, or mixtures thereof, together with a water-soluble, nutritionally acceptable carrier, such as mineral components, vitamins, carbohydrates, fats and proteins. All such components are supplied in a dried form, if and when the beverage is provided in a dry form. The beverage supplied in the form ready for consumption additionally contains water. The final solution of the beverage may also have a controlled tonicity and acidity, e.g. as a buffered solution, in accordance with the general suggestions provided in the paragraph above.

The pH remains preferably within the range from 2-5, especially 2-4, for the prevention of bacterial and fungal growth. A sterilized beverage may also be used, with pH of approximately 6-8.

The beverage may be supplied alone or in combination with one or more of therapeutically effective compositions.

The use of L. reuteri DAN080 cultures, partially inactivated L. reuteri DAN080 cultures, liquid supernatants of L. reuteri DAN080 cultures, concentrated supernatants of L. reuteri DAN080 cultures, and dried supernatants of L. reuteri DAN080 cultures and other above-mentioned forms of antimicrobial agent of the present invention for manufacturing a composition for prevention, alleviation or treatment of gastritis and other related diseases, e.g. gastric and duodenal ulcer, gastric and duodenal cancer, intestinal disorders, GIT disorders and diarrhoea.

Further aspects of the present invention cover uses where the composition is a pharmaceutical composition. This pharmaceutical composition may include pharmaceutically acceptable carriers and/or additives, such as diluents, preservatives, solubilizing agents, emulsifiers, adjuvants and/or carriers useful in the methods, the use of which has been disclosed herein in accordance with the invention.

Furthermore, as used herein, ‘pharmaceutically acceptable carriers’ are well known to skilled artisans and may cover, but are not limited to, 0.01-0.05 M phosphate buffer or 0.8% saline. In addition, such pharmaceutically acceptable carriers may be aqueous or non-aqueous solutions, suspensions and emulsions. Examples of non-aqueous solvents are propylene glycol, polyethylene glycol, plant oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's solution with dextrose, dextrose and sodium chloride solution, Ringer's solution with lactates or plant oils. Preservatives and other additives may also be present, such as antimicrobials, and antioxidants, chelating agents, inert gases, and the like.

Still further aspects of the present invention cover uses wherein the composition is a dietary supplement and/or a component in the form of solid food and/or beverage. Such a composition of the invention, such as a pharmaceutical composition or that supplied with food or feed, may optionally contain a carrier and/or a certain amount of a second or subsequent active component, exerting an effect on gastritis and other related diseases, e.g. gastric and duodenal ulcer, gastric and duodenal cancer, intestinal disorders, GIT disorders and diarrhoea.

The improvement in and prevention of gastritis and other related diseases, e.g. gastric and duodenal ulcer, gastric and duodenal cancer, intestinal disorders, GIT disorders and diarrhoea, takes place as a result of administration of compositions based on L. reuteri DAN080 cultures of, partially inactivated L. reuteri DAN080 cultures, liquid supernatants of L. reuteri DAN080 cultures, concentrated supernatant of L. reuteri DAN080 cultures, and dried supernatants of L. reuteri DAN080 cultures, and other above-mentioned forms of the antimicrobial agent of the invention. The therapeutically effective amount is approximately 0.0001-0.2 g/kg of body weight per day.

Target Groups for Administration:

As will be readily understood by one skilled in the art, the new therapeutic and prophylactic agent, methods of use of the same and pharmaceutical compositions according to the present invention are particularly suitable for administration to humans, domestic animals, pets, animals involved in sports, broilers, laying hens, mice, rats, guinea pigs, rabbits, and other animals, e.g. laboratory animals, including the primates, wild animals (living in and/or outside zoos), irrespective of age.

Launching of salami sausage or other marketable product is being considered, the product showing special health promoting properties, and being prepared by a the technological process involving lactic fermentation bacteria, making use of the following:
1) bacteria of the genus Lactobacillus—especially L. reuteri DAN080, showing specific probiotic characteristics;
2) technique which would allow preserving in the product: salami sausage (or others), live bacterial cultures. The products of fermentation of these bacteria ensure an excellent flavour quality of the product, and simultaneously, maintain bacterial homeostasis of the gastrointestinal tract of a consumer.
Due to their specific antibacterial properties, the fermentation products:
reduce colonization by pathogenic bacteria, including H. pylori,
protect against infections of the gastrointestinal tract,
alleviate the course of infections, among others H. pylori infections
prolong the expiry date of the product
when used for coating of sheathing, protect the product against contaminations, while preserving the same.

Generally, the term “probiotics” refers to such microorganisms which have been commercially used as additives to food and feed, and also have found their use in the pharmaceutical industry. The definition of ‘probioticum’ comes from the middle 1970s, when selected microbes were used in the feeding of animals.

Probiotics enhance public health conditions, which enhancement results mainly from protection of the population against systemic diseases and infections, and also from weakening of the symptoms or reduction of effects of such systemic diseases. Therefore, studies on probiotics concern the assessment of the scope within which the specific bacterial strains prevent the occurrence of disorders, and also explain the circumstances of their health promoting effect.

The alimentary system, being a highly organized ecosystem, due to protective properties of the intestine microbiota, intestine mucosa and the immune system, provides an effective barrier against pathogenic microorganisms. Probiotics enjoy a great interest in supplementary therapy in the states of bacterial gastro-intestinal infections. At present, intensive research is being conducted in many centers concerning H. pylori infections, which are clinically manifested mainly by gastric and duodenal ulcer, and contribute to the development of gastric cancer. Considering the fact of epidemiological spread of H. pylori infections in various regions worldwide, it becomes important to find methods which would limit the process.

The health promoting activity of lactic acid bacteria consists mainly of prevention of colonization of the mucous membranes (e.g. of the intestine) by undesirable microbiota, while the products secreted and released by the bacteria to the extracellular surroundings, such products as active compounds: acids, hydrogen peroxide (protons), enzymes, bacteriocins, or bacterial degradation products: fragments of the bacterial wall, affect the growth of other microorganisms (including pathogenic) and thus harmonize the function of the alimentary system. Post-fermentation products of lactic acid bacteria metabolism possess also a capability of decreasing the level of bacterial toxins and micotoxins (fungal metabolites).

The bacteria L. reuteri DAN080 are also characterized by the resistance to the effect of thermal stress occurring during the fermentation process. The production of lactic acid, the agent providing products with sensory qualities, should be maintained by bacteria at a relatively constant level, irrespective of the temperature in which the fermentation develops and the product is stored.

The unique bacteria of the genus Lactobacillus—L. reuteri DAN080, are intended, among other things, to be marketed as a probiotic in the production of cold cuts of meat. The results of preliminary studies with the use of an animal model show that, following an oral administration of the fermentation products of these microorganisms to the infected (with H. pylori and other bacteria causing gastrointestinal tract infections) stomach of a mouse, the clear reduction of the infection occurs. The studies show that thermostable fermentation products of the bacteria L. reuteri DAN080 improve the immune condition in infected mice, and at the same time, protect the stomach against further colonization. The observations supported by the in vitro studies allow the speculations that a decrease in the distribution of H. pylori as a result of the activity of fermentation products of the bacteria L. reuteri DAN080, may lead to a similar effect in humans at risk of gastric ulcer, or patients with ulcerative gastritis and duodenitis and other infections of the gastrointestinal tract.

The characteristics of cellular metabolites observed in laboratory conditions enable the assumption that L. reuteri DAN080 may be used in the technology used in production of salami sausage and other food products and beverages.

It is anticipated that the life functions of lactic fermentation bacteria L. reuteri DAN080 will be maintained after the manufacturing of salami sausage (or other products). It is also anticipated that the innovative fermentation process will increase bioavailability of macro- and microelements from such a salami sausage, primarily of calcium and magnesium. It should be emphasized that the presence of live bacteria will considerably extend durability of the salami sausage (or other products) without the addition of artificial preservative agents.

It should also be indicated that to-date, on the market of food products, despite the existing traditions, there is a lack of production of this type of salami sausage with the ability of harmonizing the function of the intestines and improving the digestion process of consumers.

At present, it has been found that the changes of nutritional habits and the accompanying enzymatic deficiency on the level of gastrointestinal tract (enzymes) are accompanied by unexpected processes of regrouping of natural microbiota, with respect to the composition and amount of the autochtonic inhabitant microorganisms. In place of the natural, innate microbiota of a healthy host, or a host with no relapse of gout attack the mucosal surfaces are colonized by other bacterial species, with various intensity, different from the natural. These ‘new’ microorganisms, while settling new ecological niches, reveal their own virulence factors. Then, according to the degree of virulence, a local/systemic infection and local or systemic inflammation develop. These processes take place mainly in the gastrointestinal tract and urogenital system.

Role of Vitamin D:

In gout patients, the blood level of 1.25(OH)2-vitamin D3 is significantly lower (p<0.05), when compared to the concentration of this vitamin in healthy individuals (8.8 mg/dL+/−0.2 vs. 5.6+/−0.2 mg/dL), whereas among males suffering from gout and those free from podagra no differences are observed in the level 25(OH)-vitamin D3. It is thus clear, that in gout patients, uric acid per se may directly reduce the level of 1.25(OH)2-vitamin D3 in blood by inhibiting 1-hydrolase activity (see: Takahashi S, Yamamoto T, Moriwaki Y, Tsutsumi Z, Yamakita J, Higashino K. Decreased serum concentrations of 1,25(OH)2-vitamin D3 in patients with gout. Adv Exp Med Biol. 1998; 431:57-60).

The role of vitamin D and its active metabolites in the defense responses of the organism covers several levels.

On the first level are epithelial cells which constitute a physical barrier protecting against injury and/or infection/invasion. Active hormone 1.25(OH)2 vitamin D enhances the physical barrier by stimulating genes encoding gap junction proteins, adherence genes, tight junction genes, and enhances intercellular communication (proteins: connexin 43, E-cadherin, occludin).

Secondly, vitamin D has a stimulatory effect on epithelial cells in the synthesis of antimicrobial peptides of innate immunity, including beta-defensins, cathelicidin LL-37.

Subsequently, vitamin D stimulates expression of potentially active antimicrobial peptides synthetized in macrophages/neutrophils, and increases the potential of oxygen burst in macrophages.

Besides, it enhances the neutralization of endotoxins through LL-37.

With respect to acquired immunity, vitamin D shows a suppressive effect, manifested as its capability for the inhibition of T lymphocytes proliferation. It exerts a suppressive effect on immunity dependent on the production of cytokines and immunoglobulins through activated B lymphocytes. It inhibits the activity of Th1 lymphocytes, and reduces synthesis by Th1 IF-gamma and IL-2 (stimulator of antibodies and cytokines). These lymphocytes participate in the development of disorders of the autoimmune background (e.g. type 1 diabetes, rheumatoid arthritis, autoimmune inflammation of the intestines, and multiple sclerosis).

The Role of Vitamin E:

A relationship was shown between a diet, and more precisely between higher consumption of meat and total proteins and decreased consumption of fruits, vegetables and vitamin C, and a risk of gout development. It was confirmed earlier that red meat, fruits of the sea, beer and high proof alcohol, and also total protein, wine and vegetables rich in purines increase a risk of gout development; while recently, dairy products have been identified as protective agents. Clinical studies in humans showed that vitamins of anti-oxidative activity (vitamin E, vitamin C, beta-carotene, vitamin A) do not significantly inhibit the process of osteoarticular inflammation of the knee, as has been previously suggested. However, in view of the fact that diet is inevitably commonly available, it is considered that even a slight improvement of health, which is the result of nutritional changes, may lead to a great effect on the health of the population. Since there is evidence showing the effect of nutritional factors on, among other factors, the course of podagra, they should not be belittled, but commonly popularized (see: Choi H K. Dietary risk factors for rheumatic diseases. Curr Opin Rheumatol. 2005; 17(2):141-6).

In accordance with the present invention, vitamins D and E are used in the form of commercially available nanoparticles. Nanoparticles of vitamins D and E are characterized by higher bioavailability. However, following an oral administration, the measurements of the levels of these vitamins in blood, performed within the standard periods after administration show a normal or only slightly elevated values. The measurement performed within a time shorter than the standard ones confirms an elevated bioavailability of vitamins D and E.

It is known that in the course of gout, the intestinal tract enzymes decomposing food rich with proteins and purine compounds do not function properly.

In the application No. WO 1988/008450—“Gene therapy for metabolite disorders”, it is only shown that a new therapy is possible, the therapy being based on recombinants for the treatment and prevention of undesired conditions characterized by accumulation or increased concentration of metabolites. The inventors of the solution propose the use of recombinants producing oxalate oxidase and oxalate decarboxylase in order to prevent oxalic acid diathesis and the formation of kidney stones. Apart from this, a recombinant with gene encoding uricase is to be useful for metabolisation of uric acid. Such a therapy is to treat and prevent gout and formation of stones. Oxalobacter formigenes OxB (ATCC 35274)—a bacterium naturally settling in the human gastrointestinal tract, is the microorganism, being a source of the genes encoding oxalate decarboxylase and oxalate oxidase inserted into the recombinants. Uricase gene was isolated from pig's liver.

Uric Acid Metabolism:

In primates, birds and some reptiles, uric acid is the final product of purine metabolism. In the human body adenine and guanine are metabolised to xanthine. From xanthine, in turn, after oxidation with xanthine oxidase, uric acid is formed according to the reaction:

xanthine+HO+0_->uric acid+0˜

Superoxide dismutase converts superoxide anion radical (O˜) to hydrogen peroxide (Lehninger, A. L: 1975, Biochemistry, 2nd Edition., Worth Publishers, New York, pp. 740-741). Enzymes participating in the metabolism of uric acid commonly occur among mammals, excluding humans. In these animals, urates are re-adsorbed in kidney and transported to liver where, with the participation of hepatic uricase, urates are converted to alantoin soluble in water, whereas humans are genetically predisposed to the formation of kidney stones (see: Gutman A B, Yu T-F: Uric acid nepholithiasis, 1968, Am. J. Med. 45:756-779).

In accordance with the present invention, the anti-microbial agent selected from the group comprising the whole culture of L. reuteri DAN080, liquid supernatant, concentrated supernatant and dried or lyophilized supernatant obtained from the culture of L. reuteri DAN080, and from mixed bacterial cultures comprising L. reuteri DAN080, and liquid supernatants, concentrated supernatants and dried or lyophilized supernatants obtained from cultures of prokaryotic and eukaryotic recombinants and whole cultures of prokaryotic and eukaryotic recombinants, in which and/or from which genes are utilized, which genes provide specific modulatory, inhibitory, homeostatic activity against H. pylori and other bacteria; proteins/oligopeptides/peptides of the molecular weight of approximately: 150 and/or 141 and/or 115 and/or 95 and/or 90 and/or 86 and/or 83 and/or 77 and/or 71 and/or 63 and/or 59 and/or 56 and/or 49 and/or 46 and/or 43 and/or 39 and/or 34 and/or 32 and/or 30 and/or 22 kD or lower, that are purified/isolated from liquid/condensed/dried supernatant obtained from L. reuteri DAN080, and purified/isolated from whole cultures of L. reuteri DAN080, and from other mixed bacterial cultures comprising L. reuteri DAN080, and purified or isolated from cultures of prokaryotic and eukaryotic recombinants, in which and/or from genes are utilized, which genes provide specific modulatory, inhibitory, homeostatic activity against H. pylori and other bacteria, or mixtures thereof useful in prophylaxis and treatment of medical conditions developing as a result of infections caused by bacteria, fungi and other pathogens of the gastrointestinal tract, body integuments and other systems, such as the urogenital and respiratory systems in vertebrates, finds its special use with respect to gout.

The present invention is based on the use of L. reuteri DAN080, optionally in combination with other bacteria and genetic engineering products in the prophylaxis and treatment of gout.

Similar to other probiotics, the probiotic potential of the bacteria L. reuteri DAN080 is evaluated based on the possibilities of passage through the gastrointestinal tract, production of antimicrobial compounds, degree of adherence to the epithelial mucin (e.g. intestinal epithelium), production of biogenic amines, mucin degradation, drug sensitivity pattern.

During fermentation, which takes place in the gastrointestinal tract, urogenital tract, body cavities and ducts, considerable amounts of final acidic metabolites are released by bacteria, accompanied by a decrease in pH. The products are difficult to quantify and include hydrogen peroxide and diacetyl, being the agents regulating microbiological relationships in the environment (antibiosis). Bacteriocins are important in the selection of microbiota triggering the fermentation. The strain/strains were identified and characterized morphologically and biochemically, as well as molecularly (identification), with respect to capabilities of survival in an acidic environment, in the presence of bile salts, capability for the utilization of proteins, starch, fats, for production of hydrogen peroxide, for bile salts hydrolase activity, and also for the production of substances inhibiting the growth of other bacteria undesirable in the gastrointestinal tract, and the determination of resistance to antimicrobial compounds. The evaluation shows non-infectiousness of the strain L. reuteri DAN080, which was tested on animals with impaired immunity.

Gram-positive bacteria encode proteins required for the incorporation into own cellular wall (D-alanine esters). This process, with the participation of teichoic acids, is important for the bacterial cell and its tolerance to acidic character of the environment, its resistance to antimicrobial peptides, its adhesion, formation of a biofilm, and degree of its virulence. The presence of D-alanine residues is important for the functioning of L. reuteri DAN080 cells and their survival in the gastrointestinal tract. It was found that treatment with catalase, changes of pH, and heating up to 80° C. do not affect the bactericidal activity of L. reuteri DAN080. Even the treatment with trypsin and proteinase K did not affect this characteristic. No reduction in antimicrobial activity was observed when an increased availability of glucose (source of carbon) and peptone (source of nitrogen) in the medium has occurred. The bacteria L. reuteri DAN080 survived also in pH 3 and subsequently were not sensitive to the activity of cholic acid and bovine bile, while still exhibiting bile salts hydrolase activity, and ability to produce antimicrobial compounds.

L. reuteri (LR)—including L. reuteri DAN080, are microorganisms which may produce hazardous primary and secondary metabolites, including organic acids, diacetyl, CO₂ and various antibiotic-like substances, such as reuterin, reutericin, reutericyclin, cobalamin, etc.

Some strains of LR possess a capability of synthetizing and releasing bacteriocins. One of these is reutericin 6—a bacteriocin, which shows both bactericidal and bacterioststic activity with respect to many species of bacteria, especially those Gram-positive. Lytic power was the strongest in the poorly opaque environment with a small number of live cells, in presence of beta-galactosidase (leakage from bacterial cells). This bacteriocin is not active against Gram-negative bacteria and does not occur in the strains of L. reuteri producing reuterin.

Reutericin 6 has a molecular weight of 2.7 kDa and comprises 67% of hydrophobic and polarly neutral amino acids, among which no lanthionine is found. The structure of the molecule is cyclic, and impossible to differentiate from gasericin A (similar molecular weight and amino acid sequence). Both bacteriocins differ with respect to bactericidal strength. Although they cause leakage of potassium ions from the cell and from liposomes, the strength of the leakage is different. Structurally, the bacteriocins are mainly in the form of alpha helises, with the difference in the number of amino acids with D and L configuration present. Reutericin 6 has two D-alanine residues among all 18 alanine residues present, while in gasericin there is only one such a residue. The number of amino acids residues, differing in their D or L configuration, determines the bactericidal activity of LR.

LR show anti-microbial activity, for which none of the known bacteriocins or reuterin or else organic acids are responsible. Reutericyclin shows a wide inhibitory spectrum of antimicrobial activity. Its activity does not inhibit the growth of Gram-negative microorganisms; however, E. coli mutant, with the LPS structure different from that of the non-mutant strain, is sensitive to the effect of reutericyclin. Reutericyclin acts against cells in a dose-dependent way. It does not destroy spores, but violates the conditions in which the germination of spores occurs. The addition of fatty acids to the bacterial culture medium changes the activity of reutericyclin. Reutericyclin—as a molecule, is hydrophobic, has a negative charge, and molecular weight of 3.49 kDa. Structurally, reutericyclin is a derivative of tetram acid (see: A. Höltzel, M. G. Gänzle, G. J. Nicholson, W. P. Hammes, and G. Jung, Angew. Chem. Int. Ed. 39:2766-2768, 2000).

Reuterin production is enhanced in the presence of glycerol.

Reuterin is a substance of antimicrobial activity produced mainly by L. reuteri during the process of anaerobic fermentation in the presence of glycerol. Maximum production of this substance occurs in the static phase and the phase of logarithmic bacterial growth.

In the presence of glycerol, L. reuteri synthetizes β-hydroxypropanal (HPA) which is subsequently secreted into the medium. It was confirmed that in a water solution reuterin occurs as a mixture of three forms of β-hydroxypropanal: monomeric, hydrated and dimeric, which remain in balance.

This compound was first isolated, purified and identified by Talarico and Dobrogosz. To-date, a number of reuterin properties have been demonstrated, primarily it is an effective inhibitor of the growth of a wide spectrum of not only bacteria, but also of fungi and protozoa. The mechanism of reuterin activity has been investigated for over 20 years, and at present it is known that this compound may exert an effect on microorganisms in a dual manner. The substance may inhibit the activity of bacterial ribonucleotide reductase (an enzyme catalyzing the first stage of DNA synthesis) by competing with ribonucleotides for binding sites in the DNA sequence, or by reaction with unstable thiol groups of this enzyme. In addition, it was found that reuterin may enter into direct reactions with thioredoxin, a protein performing the role of a reducer of many enzymes, including ribonucleotide reductase, thus inhibiting the enzymatic activity of this protein. Reuterin is a substance soluble in water, acting within a wide pH values, resistant to lipolytic and proteolytic enzymes treatment. The optimum conditions for the growth and production of reuterin by L. reuteri is the temperature of 37° C. and pH 4.6-5; this compound also remains stable in an environment of a considerably lower temperature and acidity of the medium.

The strains of LR also produce cobalamin (vitamin B12) in the process of cofermentation of glycerol and glucose.

Genetics: It is possible to construct a shuttle vector (e.g. Escherichia coli-lactobacillus) transferred into L. reuteri DAN080 cells, so that the transformant obtained showed its activity, e.g. antimicrobial.

There is a possibility of cloning genes in L. reuteri DAN080, (known is the cloning of beta-galactosidase heterodimer in L. reuteri cells other than L. reuteri DAN080 cells), and it is assumed that the expression of such structural genes must be associated with the activity of proteins involved in maturation (cutting, cyclic form) and secretion outside the L. reuteri DAN080 cell by various transporting systems. It is also assumed that in L. reuteri DAN080 the mechanisms significant for the autoprotection of cells against such strong inhibitors as e.g. reutericin 6, are present.

It is possible to construct with L. reuteri DAN080 cells a ligated gene of the green fluorescence protein into the secretion vector which generates release of a chimeric protein capable of glittering (marker).

It is possible to adapt L. reuteri DAN080 cells to be incorporated into the Nisin-controlled gene expression (NICE) system by ligating nisA promoter (PnisA) and nisRK DNA fragments into the shuttle vector E. coli-L. reuteri pSTE32. In such a chimeric plasmid the expression of heterologic genes is possible with the induction of nisin.

As regards catheters according to the present invention, two types of polymers were used as a basic material for manufacturing the catheters: PVC and silicon.

PVC is a cheap polymer, the safety of which has been confirmed for many years. At present, a new generation of plasticizers is used, additionally increasing safety of PVC. Silicon is an expensive polymer; however, it is characterized by a very high compatibility.

According to the invention a catheter made of PVC or silicon is coated with a polymer nanocoating made of polyvinyl pyrrolidone (PVP). Only in contact with water PVP forms a thick jelly solution which lubricates the polymer surface.

Polymer coating made of PVP may also be applied on catheters made of polyurethane or natural latex.

PVP is widely used in pharmacology for the production of biomaterials intended for contact with blood, in results of its biocompatibility (lack of toxic effect, including degradation effect on blood cells hemolysis, lack of effect on the host immune system). An advantage of nanocoating made of PVP is that this coating is resistant to activity of microorganisms, including pathogenic organisms.

The only drawback of such coating is the need for wetting the catheter prior to insertion the procedure; however, this problem can be easily solved in accordance with the invention inside the package, when using a kit for catheterization according to the invention.

According to the invention, nanocoating made of PVP is chemically bonded to a polymer of antimicrobial activity, such as chitosan salts. This is a polymer obtained from Crustacea shells. Chitosan salts are known for their use in medicine. They are safe, bioavailable and biodegradable.

In order to prove an unexpected synergism, the coatings made of chitosan salts and PVP were examined separately and in combination. In addition, the composition of individual coatings was enriched with other active substances increasing the spectrum of activity of the surface of the catheter according to the invention, such as extracellular metabolites of L. reuteri DAN080 (deposit DSMZ—access number—DSM 15693—in accordance with a Budapest Treaty on the International Recognition of the Deposit of Microorganisms for the Purposes of Patent Procedure, on 20 Jun. 2003), and vitamin D in the form of nanoparticles and/or nanoparticles of silver.

Forming of solid polymer coating may be proven by two methods—physical and chemical, based on covalent polymer chains bonded through covalent bonds. Physical anchoring of polymer chains can be achieved by forming a nanocoating of discontinuous PU layer and application of PVP solution.

Due to London forces, the PVP chain is partially immersed in the basic polymer and partially protrudes therefrom. This nanocoating, having thickness of about 50,000 C—C bonds (10 nm), when immersed in water forms a sort of brush with excellent lubricative properties. This original technology has already been developed.

Alternatively, on the surface of the basic polymer, a desired layer is deposited by the method of forming free radicals by hydrogel absorption. Such free radicals are very active and easily ‘catch’ other chemical substances forming stable covalent bonds.

The application of both technologies is possible, as both of them provide the desired coatings.

Nanocoatings obtained on the polymer surface are tested to evaluate their biocompatibility, development of a biofilm and colonization by microorganisms. Their friction coefficient against pig tissue is also examined. The evaluation is performed making use of a specially constructed device, improved to meet the needs of the current invention. The optimum friction coefficient ensures painless insertion of the catheter, however, without any risk of its slipping out.

The novel properties of the external nanocoatings of the catheter according to the invention were achieved by physical and/or chemical bonding of active agents.

An active agent of the external catheter nanocoatings is the component originating from the new DAN080 strain of lactic acid bacteria L. reuteri identified by the present inventor, deposited on Jun. 20, 2003—in accordance with a Budapest Treaty on the International Recognition of the Deposit of Microorganisms for the Purposes of Patent Procedure, in DSMZ collection—Deutsche Sammlung von Mikroorganismen and Zellkulturen GmbH, in Braunschweig, DE, access number: DSM 15693.

It has been now unexpectedly found out that extracellular metabolites of L. reuteri DAN080 are a desired component of the external nanocoating, or one of the layers which coat the catheter according to the invention.

After the specified period of growth, the L. reuteri DAN080 culture is centrifuged, and the liquid, concentrated supernatant and dried supernatant is the product of specific capabilities and activity with respect to the regulation of bacteria growth in vitro and in vivo. After the electophoretic separation performed using a liquid supernatant, concentrated supernatant and dried supernatant collected at a specific time, specific proteins were visualized, the proteins having molecular weight within the range of 150-22 kD and smaller, which are responsible for homeostasis and bacteria growth regulation in the patient's body. These proteins, both in an isolated and purified form, and also in the form of a liquid supernatant, concentrated supernatant and dried supernatant, collected after the proper period of culturing the L. reuteri DAN080 culture, independently or in a mixture with other lactic acid bacteria isolated by and being the property of the present inventor Danuta Kruszewska, confer new, unexpected properties to nanocoatings coated on the catheter according to the invention, which properties increase the safety and comfort for patients, while showing homeostatic and regulatory effect on bacterial growth on the surface of the catheter according to the invention.

Extracellular metabolites of the L. reuteri DAN080 are used in combination with other known agents of antibacterial activity, according to the present disclosure. The methods of isolation, culturing the L. reuteri DAN080 bacteria and collection, isolation and purification of extracellular metabolites of the L. reuteri DAN080 are disclosed in a parallel patent application claiming the same priority date as the present invention.

Apart from extracellular metabolites of the L. reuteri DAN080, vitamins are also used as active agents, especially vitamins D and E in the form of nanopowders, in order to enhance the patient's immune mechanisms, and also as agents reducing the growth of microorganisms and the formation of biofilm according to the present invention nanoparticles of silver, small dicarboxylic acid and chitosan salts may be used. In order to gradually release the above-mentioned active agents, known compositions for delayed release were used, for example compositions soluble in physiological fluids for coating the hydrogel. The selection of the used technical means for delayed release of the substances depends on the necessity of preserving their properties for the time adjusted to the anticipated period of catheter placement inside patient's body, considering the diffusion coefficient and/or degree of bonding on the surface.

Description of Experiments

Preparation of chitosan salts: The raw material used for producing proper chitosan salts is technical chitin obtained from Basinomycetes (Lentinus edodes, Le 323), according to the publication No. PL 384836—“Method for obtaining fungal chitosan’ of 12 Oct. 2009, or from the scutum of Antarctic krill (Euphausia superba). The residues of organic and inorganic contaminants are removed from the polymer in the processes of demineralization and deproteinization. A part of the chitin thus obtained is subjected to the process of chemical decomposition in order to reduce its polymerization degree to the desired level. This allows obtaining chitosans with similar deacetylation levels and different molecular weights. Chitosan is obtained in the process of alkaline deacetylation. Its properties are modified by changing the reaction time and temperature. Salts are obtained in the reaction of chitosan with organic acid(s) in aqueous environment. Thus obtained salt solution is lyophilized. The evaluation of the properties of raw material and products is monitored.

In order to obtain (in laboratory conditions) chitosans of various molecular weights and deacetylation levels the following procedure is applied:

preparation of the raw material for the production of chitosan: technical grade chitin is purified, the molecular weight of the polymer is modified in order to obtain chitin having various polymerization levels. Chitosan of the required properties is obtained through control of parameters of the deacetylation process.

Chitosan salts obtained in laboratory conditions are tested for their antimicrobial properties.

The biological testing of chitosan salts that facilitates quick assessment of their antimicrobial activity, allows monitoring, control and selection of the optimum parameters of their production process, especially the ranges within which the deacetylation level and molecular weight of chitosans should be modified.

The optimization of the method for obtaining salts is carried out from the aspect of the most intensive biological activity after the sterilization process, taking into consideration the destination of the catheter and the environment in which it will be used.

Adhesion of Multi-Drug-Resistant Bacterial Strains to Coated and Non-Coated Surfaces of the Polymers Tested:

The pathogenesis of many bacteria is associated mainly with the ability of these organisms to irreversibly adhere to polymer surface and to produce an extracellular glycocalyx in the course of colonization.

The percentage of adherence is defined as a rate of CFU recovered from the polymer tested to CFU of marker bacteria (multi-drug-resistant strains) in the culture fluid.

Antimicrobial Activity of Chitosan Salts with Relation to Multi-Drug-Resistant Microorganisms:

Each sample is cultured together with one of the tested strains (CFU 10³). At various time points (0, 30, 60 and 120 min) after incubation at the temperature of 37° C. samples are collected, vortexed and placed on plates plated with agar solidified medium. The CFU values are counted after incubation of microorganisms at the temperature of 37° C. for 48 hours. The CFU counts in samples taken before incubation is used to calculate the reduction of CFU.

Studies on animals were based on the non-infectious animal model of the adult rats. Six-month-old Sprawgue-Dawley females rats were used with the weight of ±350 g (n=90). The rats were divided into 9 groups. Into the urinary tract of rats (n=36; 3 groups) polymer rods were inserted covered with antibacterial and lubricous coatings. Impregnated rods were made of PVC, polyurethane and silicon, respectively. Another three groups of animals (n=36) served as negative controls in which the rats had only uncoated rods inserted into their urethra. The subsequent two groups (n=12) served as a positive controls in which the rods inserted were coated with nanosilver and PVP. The remaining rats (n=6) did not have any biomaterials inserted.

The above-mentioned rods were placed in rats from the peritoneal cavity to the urethra. They were inserted by piercing below the exposed urinary bladder, at the site between the urinary bladder and the urethra. After drilling through a micro hole in the urethra, the rod was fixed with its rounded tip towards the external wall of the urinary bladder. The length of the rod was made so that it did not protrude from the external urethral orifice, thus enabling study of the encrustation process and to avoid the rods being pulled out or bitten by the rats. The diameter of the rod had to be twice as small as the urethra diameter, and external tip of the rod had to be rounded.

The animals remained under veterinary supervision. After 7 to 14 days after the onset of the study the rats are sacrificed. Samples of urine, blood, tissues and the rods were taken in sterile conditions. Urine and blood samples were subjected to microbiological examinations.

In serum samples, lysozyme levels and defensins activity were measured.

The surface of the rods was analysed for the colonization by microorganisms and the degree of incrustation by glycocalyx.

Isolated organisms adhering to the surface of the rods were identified and their biochemical activity characterized, including the determination of their sensitivity to antibiotics.

Prior to fixation, the tissues were analyzed for the settlement of microorganisms. Fixed samples of tissues examined morphologically and immunochemically and the presence of defensins was determined.

Characteristics of Microorganisms Isolated from Animal Tissues:

It was found out that the microorganisms were identical as the strains tested (antibiogram, integrons profile, urease activity in consent). As bacteremia/bacterinuria related with the polymer of which the rod was made, and its layer in contact with the site of activity was considered the state when the microorganism was characterized by antibiogram, integron profile, and urease activity, identical as those of the microorganism isolated from the tip or other segment of the catheter, from urine and blood of the animals in the study.

Quantitative Determinations

Bacteria Isolated from Blood, Urine, Tissues and Polymer Rods were Counted by Classical Methods:

Serial dilutions of the animal fluids or homogenized tissue (urethral bioptates) were plated on the appropriate media and cultured at the temperature of 37° C. for 24 hours, in aerobic and anaerobic conditions. The number of bacteria was counted and calculated for 1 ml of blood/urine or 1 g of tissue as the mean value obtained from the 3 tests performed for a bioptate taken from a single animal. The obtained segments of polymer rods were cultured and counted by means of a quantitative technique. The samples were incubated on a solid medium (5% sheep blood agar) or on another growth medium, and the colonies cultured were counted after 24 h incubation at 37° C.

Bacterial Identification System:

Identification of bacteria was performed on the basis of based on biochemical activity characteristics of isolates, usually using detection systems API (bioMerieux, France), RT PCR.

Virulence Characteristics of Isolates

Quantitative Analysis of Activity Urease of Ureolytical Bacteria In Vitro:

Quantitative analysis of the bacteria examined was performed at various pH. The rate of urea conversion to ammonia was measured according to manufacturer recommendations (Wako Chemical). The urease activity was then expressed as μmol urea hydrolysed after 1 min, 1 mg of protein. A standard curve was obtained with NH₄Cl within the range 0.1-20 mg N—NH₄⁺/L.

Antimicrobial Susceptibility Profile as a Marker for Detecting Similar Strains

The minimum inhibitory concentration (MIC) was determined. Bacteria were tested using the disc diffusion method on Mueller-Hinton agar, according to CSLI guidelines. In both assays, E. coli ATTCC 25922, P. aeruginosa ATTC 27853, S. aureus ATTC 29213, E. faecalis ATTC 29212 were used as the reference strains for antimicrobial susceptibility testing.

Presence of Integrons/Transposons PCR

The phenomenon of increasing selective resistance in bacteria, resulting from ineffective treatment, is one of the biggest challenges for health care. Mobile integrons are one of the mechanisms of spreading multi-drug-resistance. Integrons are able to transfer within the bacterial genome, as well as horizontally to and from integron-positive cells. In the study, the integron pattern was determined in bacterial strains isolated from the tested samples (urine, blood, tissues, polymer rods). Resistance genes localized in the integron box were analyzed by means of PCR with specific primers.

Integron profiles are useful tools in comparing isolates which are considered as being identical.

Effect of New Biomaterials on Innate Immunity Response:

In natural physiological conditions the urinary tract is partially sterile. This phenomenon is related mainly to innate immunity response, and particularly to antimicrobial substances, such as defensins, cathelicidins, lactoferrin and lysozymes. Defensins and lysozyme are the best known and the most important for urinary tract immunity. Lysozyme levels were measured in serum, urine and tissue samples using the ELISA test, and enzymatic activity measured using the turbidimetric method. Selected defensins were measured in homogenized tissues using a sandwich ELISA test. Test RT-PCR is performed to analyze the expression of defensins in the cells of rat populations.

Measurement of Cytotoxicity of New Biomaterials:

It is of the utmost importance that the material from which the inserted catheters are manufactured is biocompatible, therefore, different types of surfaces and surface coatings were examined.

A number of tests for compatibility are used which allow to determine whether a structure and/or a particular coating activates the innate immune system, if the tested materials induce necrosis and/or apoptosis, if the material is cytotoxic, and if it interferes with cell proliferation. The tests are performed both in vivo and in vitro. The results of the above-mentioned tests constitute a basis for the selection of a proper material for catheter nanocoating, which material, as such, satisfies the condition of being nontoxic, inducing minimum cell death, causing no or only limited inflammation, and evoking no overt inflammation. Throughout the investigations different materials for catheter production were tested, alone or in combination with the catheter.

Several tests were applied to assess the performance of biomaterials:

1) in conformity with established (external) standards, based on the guidelines laid out in FDA Modified [ISO] Matrix (Blue Book Memorandum # G95-1, Attachement A);
2) standardized testing defined by the owner of the present invention, and 3) scientific tests aiming to extend knowledge concerning the response of the organism to the nanostructures under development.

Catheter Insertion Tests:

In order to monitor the toxic effect of materials leaking from the nanocoating (urethral catheters coated in biomaterial) two rabbits are tested for each of the biomaterials used in uncoated and coated urethral catheters for 1, 4 and 12 weeks. In brief, 4 strips of nanostructure coated or uncoated material used for catheters are inserted, respectively, into the left and right paravertebral muscles using a trocar. The rabbits are monitored for toxic response, and macroscopic evaluation of the implant site carried out periodically during the experiments.

At the end of the experiment, the animals are sacrificed, macroscopic evaluation of the implant site is performed, and a photographic record is taken for subsequent evaluation. Samples of blood and muscles are collected from the site of material implantation, and frozen or fixed in 4% paraformaldehyde for histopatologic examination. Muscle tissue is processed and embedded in paraffin, and sections are prepared and stained, and tissue sections evaluated for inflammation, necrosis, fibrosis and other indicators of a toxic interaction between muscle tissue and tested material. The effectiveness of the use of extracellular metabolites of the bacteria L. reuteri DAN080 as a basic component of catheter's external nanocoating is justified by the following findings.

The experiments were performed on the effect of live L. reuteri DAN080 bacterial cultures, heat inactivated L. reuteri DAN080, and chitosan alpha-ketoglutarate on the immune system of laboratory animals.

Forty eight 2-month-old Sprague Dawley female rats with a weight of 140-275 g, were fed with feed adequate for the age of the animals, and watered ad libitum. Three days prior to experiment, all animals had catheters inserted in the jugular vein. The study was started by taking blood samples from the rats. Subsequently, the animals were administered intragastrically, by means of a gastric tube, 0.5 ml of the following preparations: suspension of the bacteria L. reuterii DAN080 live and dead cells, chitosan AKG suspension, saline. Hundred twenty min. after the first blood taking the second blood sample was taken from the animals. On the second day, the rats received the same preparations once daily for 7 subsequent days. Twelve rats received live bacteria at a dose of 10⁶ cells suspended in physiological saline. The following 12 animals also received for 8 days, 10⁶ each of thermally killed cells L. reuterii DAN080. To the next 12 rats chitosan AKG suspension was administered, and the fourth group of animals (n=12) were administered intragastrically for 8 days, at each time 0.5 ml physiological saline. On day 8 after the final dose of the preparations administered intragastrically, blood was taken from the animals from the jugular vein. For the second time on the same day, blood was taken from all rats 120 min. after the first blood taking.

Determinations of the lysozyme activity in the blood were performed in the presence of a suspension of Micrococcus lysodeikticus cells of specified density, based on the absorbance value, and comparing this value with the absorbance curve plotted from a number of standard dilutions of crystal lysozyme (Sigma-Aldrich) in PBS and suspension of M lysodeikticus cells of specified density. After 15, 30, 45, 60 min., incubation absorbance was measured at the wave length of 540 nm.

The results obtained concerning the activity of lysozyme (U/L) in the blood of rats following the intragastric administration of the tested substances, presented in FIG. 3, confirmed the stimulation of the rat immune system by live and thermally killed L. reuteri DAN080, and by chitosan alpha-ketoglutarate AKG.

Lysozyme is a hydrolytic enzyme released by certain phagocytes, such as macrophages and multinuclear leukocytes, plays a significant role in the control of pathogenic microorganisms. Lysozyme is also produced by Paneth cells located in the lining of the intestines. Lysozyme is especially active against Gram-positive microorganisms. Phagocytic activity of cells involves degradation, with the participation of lysozyme, of the cellular walls of bacteria, more precisely a cleavage of glycoside bonds in peptidoglycans. An elevated lysozyme activity induced by the introduction of metabolites of L. reuteri DAN080 bacteria stimulated the phagocytes activation or antigen presentation to phagocytes. In this way, the function of the immune system was enhanced, mainly non-specifically. This confirms that both the live and dead cells of L. reuteri DAN080 show the ability to act against many pathogens. The cells as such are not recognized as dangerous by the immune system of the organism. Antimicrobial activity of the bacteria L. reuteri DAN080, their extracellular metabolites and chitosan alpha-ketoglutarate, is additionally enhanced, because neither live L. reuteri DAN080 nor their metabolites or chitosan alpha-ketoglutarate are sensitive to the activity of lysozyme, and are not hydrolyzed in contact with lysozyme.

Hyperurikemia was induced in healthy rats by blocking the activity of urate oxydase (EC 1.7.3.3) by the inhibition of purine metabolism. After a month of feeding with an addition of an inhibitor (oxonic acid, uric acid at a daily dose of 0.4 and 0.6 g, respectively), sand and stone developed in the animal's kidney (see: Bluestone R, Waisman J, Klinenberg J R. Chronic experimental hyperuricemic nephropathy. Lab Invest. 1975; 33(3):273-9). This model serves for the testing the efficiency of functioning the catheter according to the invention. The catheterization of the kidney protects against the crystallization of stones.

The facts already mentioned above justify including vitamin D in nanocoatings. The role of vitamin D and its active metabolites in the defense responses of the organism covers several levels. On the first level are epithelial cells which constitute a physical barrier protecting against injury and/or infection/invasion. Active hormone 1.25(OH)2 vitamin D enhances the physical barrier by stimulating genes encoding gap junction proteins, adherence genes, tight junction genes, and enhances intercellular communication (proteins: connexin 43, E-cadherin, occludin).

Vitamin D has a stimulatory effect on epithelial cells in the synthesis of antimicrobial peptides of innate immunity, including beta-defensins, cathelicidin LL-37.

Subsequently, vitamin D stimulates expression of potentially active antimicrobial peptides synthetized in macrophages/neutrophils, and increases the potential of oxygen explosion in macrophages. Besides, it enhances the neutralization of endotoxins through LL-37.

With respect to acquired immunity, vitamin D shows a suppressive effect, manifested as its capability for the inhibition of T lymphocytes proliferation. It exerts a suppressive effect on immunity dependent on the production of cytokines and immunoglobulins through activated B lymphocytes. It inhibits the activity of Th1 lymphocytes, and reduces synthesis by Th1 IF-gamma and IL-2 (stimulator of antibodies and cytokines). These lymphocytes participate in the development of disorders of the autoimmune background (e.g. type 1 diabetes, rheumatoid arthritis, autoimmune inflammation of the intestines, and multiple sclerosis).

The presence of vitamin D at the site where the mucous membrane in contact with the catheter has an ability to produce antimicrobial peptides, has a stimulatory effect on antimicrobial activity of the epithelial cells lining the lumen of the urogenital system, digestive system, genital tract, respiratory system, blood and lymphatic vessels. In the case of LPS secretion by Gram-negative bacteria (causing mainly the infections of the urogenital system), vitamin D reduces the toxic effect of endotoxins by stimulation of innate immunity effector cells, which enhances the production of antimicrobial peptides neutralizing LPS.

On the other hand, vitamin D protects against allergic reactions, which may be induced by the insertion of a catheter into a site of its use.

The experiments mentioned below confirm the positive effect of the addition of extracellular metabolites of L. reuteri DAN080.

Behavioral tests were performed based on an open field test for the assessment of anti-anxiety effect, for the analysis of locomotor and exploratory activity under the influence of killed and live L. reuterii DAN080 cells on laboratory rats.

It was possible to determine the effect of those preparations on the general profile of the behavior of the animals.

Three groups of animals were administered heat treated and live L. reuterii DAN080 cells for three months at a dose of 10⁶ and saline at a volume of 1 ml intragastrically, using a tube. Starting from the second month of the experiment, the dose was doubled, and divided into the administration of the preparation in the morning and in the evening. Behavioral tests were performed 3 times at monthly intervals.

1. Open field test was performed 3 times, in the first, second and the third month of the experiment. The test was performed in a plastic box of the size 100 cm×100 cm×40 cm (height of the wall). The square floor of the box was divided by lines into 25 equal squares. The testing was performed in conditions of a quiet and bright room. Individual behaviors of the rats were observed. Each rat in the experiment was taken out of its cage and placed in the centre of the box floor.
a. The number of squares which the rat passed during 3 min. of observation was registered.
b. In the same box and under the same conditions an experiment was performed on the rats consisting of a 3 min. observation of the number of withdrawals of the animal's body.
c. In the same box and under the same conditions an experiment was conducted on the rats consisting of a 3 min. observation of the number of occurrences of the snout washing and cleaning the fur.

Horizontal activity of rats was measured by the number of traversed squares. The young rats, after one month of administration of the tested preparations, showed high locomotor activity. The decrease in this horizontal activity was observed between the second and third month of the study. The least mobile, compared to the control animals, were the rats which were administered live L. reuteri DAN080 bacteria, followed by those receiving heat treated L. reuteri DAN080 (**p<0.5, Student's t-test, *p<0.5, t-test).

During the experiment, all animals with the lapse of time and—most probably, with ageing showed a tendency towards a progressive decrease in horizontal and vertical activity.

Vertical activity is measured by the number of occurrences of the animal body withdrawals.

During the first month of the administration of the preparations the young rats were mobile, and those administered live L. reuteri DAN080 cells for at least 3 months showed a statistically significant difference in activity, compared to the control group (*p<0.5, t-test). Also, compared to the control group, a statistically significant decrease in vertical activity was noted in the rats which for 2 months had received dead L. reuteri DAN080 cells—(*p<0.5, t-test).

In the group of animals which for 2 or 3 months received live and heat treated cells, statistically significant differences were observed (*p<0.5, t-test) in the number of occurrences of the snout washing and fur cleaning, compared to the same activity performed by the control group of animals receiving exclusively saline.

In the control group, a tendency was noted towards an increase in the number of occurrences of the snout washing and fur cleaning (observation from the 1^{st} through 3^{rd} month of saline administration).

These data indicate that during the period between the 2^{nd} and 3^{rd} month of administration of the L. reuteri DAN080, the treatment exerted a calming effect on the rats. The results obtained are illustrated by FIG. 2 a-c, wherein the number of traversed squares (FIG. 7a), the number of rat body withdrawals (FIG. 7b), and number of occurrences of the snout washing and fur cleaning (FIG. 7c) are shown.

2. Open field test—social behavior. The experiment was carried out during the 3^{rd} month of the experiment, in the same box and under the same conditions. The only difference was that 2 rats coming from 2 different cages were placed in the box. The animals received intragastrically the same preparations, according to the above-mentioned schedule of division into groups. The behavior of each pair was observed for 7 min.
a. In the same box and under the same conditions an experiment was conducted with a pair of rats consisting of a 7-min. observation of the number of animals' body withdrawals.
b. In the same box and under the same conditions an experiment was carried out with a pair of rats consisting of a 7-min. observation of the number of occurrences of the snout washing and fur cleaning
c. In the same box and under the same conditions an experiment was conducted with a pair of rats consisting of a 7-min. observation of the number of occurrences of mutual sniffing.

The number of occurrences of body withdrawals, compared to the control group, showed a statistically significant decrease in the activity of animals receiving live L. reuteri DAN080 cells (*p<0.5, t-test). The statistically significant decrease in the number of the snout washing and fur cleaning, and sniffing noted in the group of rats receiving live L. reuteri DAN080 (**p<0.5, Student t-test) indicates both a lack of stressful effect and anxiety evoking in the animals after the administration of the test preparations.

At the same time, in all the experiments conducted, no statistically significant differences were noted in the frequency of defecations and urinations by the rats. This indicates a decrease in the rats' anxiety of their new surroundings and/or new conditions.

FIG. 7d-7f presents the results of behavioral tests conducted on rats receiving live and heat treated L. reuteri DAN080 at a dose of at least 10⁶ cells/ml and physiological salt at a volume of 1 ml daily. In the open field test, the social behavior of the animals was tested by examining the number of rat body withdrawals (FIG. 7d), number of occurrences of the snout washing and fur cleaning (FIG. 7e), and number of mutual sniffings (FIG. 7f).

The experiment also confirmed a stimulatory effect of the above-tested factors in contact with epithelial cells of the mucous membranes of the body cavities other than the alimentary tract, which required catheterization.

FIG. 3 illustrates the results of electrophoresis of supernatants of the L. reuteri DAN080 cultures.

### EXAMPLES

### Example 1

### Effect of Fermentation Products of L. reuteri DAN080 and Other Lactic Fermentation Bacteria on H. pylori Colonization in the Mouse Stomach

48 mice (BALB/cA) divided into 4 groups of 12 mice each were involved in the study.

The first group of mice were administered daily, by a gastric tube, for 35 days, a preparation 1 (definition 1)—0.5 ml of a mixture of neutral supernatant obtained from 10-hour culture of L. reuteri DAN080 cells in stationary phase, and other lactic acid bacteria, listed in Tables 1-4, having an anti-H. pylori activity in combination with calcium alpha-ketoglutarate (30 mM) or chitosan alpha-ketoglutarate, or others, or other alpha-ketoacids salts administered in a liquid form, or contained in bakery products or in crisps. Starting from the 11^{th} day of the experiment, the same mice were administered twice a week for the subsequent 2 weeks, 1 hour after the first treatment a portion of 0.2 ml of fresh microscopically monitored sub-culture of H. pylori cells (10⁸ cells/ml) suspended in BHI.

The second group of mice was administered daily via gastric tube, for 35 days preparation 2 (limited definition 2)—0.2 ml (10⁸ cells/ml) of L. reuteri DAN080 and other lactic acid bacteria reported in Tables 1-4 cells suspended in MRSB, or administered with bakery products or crisps, exhibiting anti-H. pylori activity in combination with calcium alpha-ketoglutarate (30 mM) or chitosan alpha-ketoglutarate, or others, or salts of other alpha-ketoacids, and subsequently, starting from the 11^{th} day of the experiment the mice were infected with H. pylori according to the infection scheme, as described for the first group.

The third group of mice was administered daily, intragastrically by a gastric tube, for 35 days preparation 3 (definition 3)—cells of L. reuteri DAN080 and other lactic acid bacteria, being the property of the inventor (10⁸ cells/ml) suspended in 0.5 ml MRSB, or administered with bakery products or crisps, having an anti-H. pylori activity in combination with a mixture of a neutral supernatant obtained from a 10-hour culture of L. reuteri DAN080 in stationary phase, and other lactic acid bacteria constituting the property of the inventor, and calcium alpha-ketoglutarate or chitosan alpha-ketoglutarate, or salts of other alpha-ketoacids.

The fourth group (positive control), twice a week for two weeks was fed by gastric tube with 0.2 ml of fresh microscopically monitored sub-culture of H. pylori cells (10⁸ cells/ml) suspended in BHI.

The results are shown in Tables 3-5.

On the 36^{th} day, all mice were sacrificed and their stomachs were examined for the presence of H. pylori in the mucosa—Table 3.

TABLE 3
Presence of H. pylori in gastric mucosa of mice from experimental
groups I-IV.

| Section of | Colonization with H. pylori | | | |
|---|---|---|---|---|
| gastrointestinal | after previous treatment as in: | | | |
| tract | Group I | Group II | Group III | Group IV |
| Stomach | − | − | − | + |

### Example 2

### Effect of Fermentation Products of L. reuteri DAN080 and Other Lactic Fermentation Bacteria on the Colonization by Ureolytic Microbiota, in Wild Animals

A diet of a group of wild animals from a zoo (n=10), without clinical symptoms indicating the disruption of the continuity of the gastrointestinal tract epithelium, constantly exposed to stress due to a rapid and permanent change of nutritional and environmental conditions, and therefore exposed to infections with ureolytic bacteria, was supplemented for 60 days, with the preparation 4 (definition 4)—being a mixture of neutral supernatant of a 10-hour culture of the cells of L. reuteri DAN080 in a stationary phase and other lactic acid bacteria mentioned in Tables 1-4, or contained in bakery products or crisps, exhibiting an anti-ureolytic bacteria activity in combination with the cells of L. reuteri DAN080 and other lactic acid bacteria constituting the property of the present inventor, exhibiting the activity against ureolytic bacteria, in combination with calcium alpha-ketoglutarate (30 mM) or chitosan alpha-ketoglutarate, or others, or salts of other alpha-ketoacids, or in combination with bakery products, crisps.

After the introduction of such additives to the diet/feed, and for a further 30 days of observations, the animals maintained good health and general well-being, with no fever, diarrhoea, or other symptoms of infection occurring.

### Example 3

### Effect of Fermentation Products of L. reuteri DAN080 and Other Lactic Acid Bacteria on the Colonization of the Skin of the Back and Face of Young Volunteers, Aged 13-17 (n=12), with the Diagnosis of Acne vulgaris, by Propionibacterium acnes

In the first group, the volunteers (n=4) were administered twice a day, for 30 days, a preparation (limited definition 5) made of a mixture of neutral supernatant obtained from a 10-hour culture of L. reuteri DAN080 cells in a stationary phase, and supernatants from the cultures of other lactic acid bacteria constituting the property of the present inventor, exhibiting anti-ureolytic bacteria activity with calcium alpha-ketoglutarate or sodium alpha-ketoglutarate or chitosan alpha-ketoglutarate, or others, or with salts of other alpha-ketoacids administered in the form of an ointment or moist compress.

The second group of volunteers (n=4) received twice a day, for 30 days, a preparation (limited definition 6) made of the cells of L. reuteri DAN080 and other lactic acid bacteria, mentioned in Tables 1-4, exhibiting anti-ureolytic bacteria activity in combination with calcium alpha-ketoglutarate or sodium alpha-ketoglutarate or chitosan alpha-ketoglutarate, or others, or with salts of other alpha-ketoacids in the form of an ointment or moist compress.

The third group of volunteers (n=4) was administered twice a day, for 30 days a preparation (narrowed definition 7) of cells of L. reuteri DAN080 and other lactic acid bacteria constituting the property of the present inventor, exhibiting anti-ureolytic bacteria activity in combination with the mixture of neutral supernatant obtained from a 10-hour culture of L. reuteri DAN080 in a stationary phase, and other supernatants from cultures of lactic acid bacteria constituting the property of the present inventor, having anti-ureolytic bacteria activity, and calcium alpha-ketoglutarate or sodium alpha-ketoglutarate or chitosan alpha-ketoglutarate, or others, or with salts of other alpha-ketoacids—administered in the form of an ointment or moist compress.

In the course of the study, in all the volunteers, healing was observed of the infected sites where Acne vulgaris occurred. No new foci of acne developed in any of the volunteers. During the 15-day observation, after termination of the administration of the preparations, no reinfection was noted.

TABLE 8
Presence of P. acnes in the skin of the back and face of the volunteers
infected (Groups: I-III).

| | Colonization by P. acnes | | |
|---|---|---|---|
| | after previous treatment | | |
| Skin | Group I | Group II | Group III |
| Back | − | − | − |
| Face | − | − | − |

It was unexpectedly found out that the preparation is effective against chronic, porous fissure-like infections with ureolytic bacteria, including infections of the feet, armpits and groin, and epidermis products, such as nails, hair, hooves and horns. The preparation protects and reduces infections in the form of abscesses and boils, as well as sycosis, exfoliative dermatitis of infants, erysipelas, impetigo contagiosa, ecthyma, folliculitis, Acne vulgaris, difficult to treat erythrasma caused by Propionibacterium minutisimum, infections of surgical and burn wounds, and bed sores. Due to its activity reducing the colonization of the skin surface and skin products, the unpleasant odour caused by changes in the pH of the skin, and the presence on its surface of odorous extracellular metabolites of ureolytic, bacteria become eliminated.

Lactic acid bacteria constituting the property of the present inventor are easily cultured on liquid or solid media MRS (de Man Rogosa Sharpe) for 24 hours at the temperature of 37° C., in microaerophilic conditions. The above-mentioned strains show characteristics which may be considered as conducive to the colonization of the gastrointestinal tract. They possess a capability of binding matrix proteins, especially collagen and fibronectin, which favours the adhesion to the epithelium of the intestine. These bacteria release proteases causing the decomposition of milk proteins, and fermentation of saccharides contained in milk, which facilitates access of the microorganisms to the nutritional substrate. Besides, for some of them inulin may be the source of carbon. Therefore, while fermenting this indigestible fructan, irrespective of other biochemical activities biased against ureolytic bacteria, the bacteria participates in the regulation of the local intestinal microbiota. All strains survive for one hour in a medium containing 20% of bovine bile, in acidic conditions, in pH of 2.5 for 2 hours, which indicates that after oral administration they may pass intact via the stomach and small intestine to the large intestine. They do not develop resistance to antibiotics and chemotherapeutics to the degree that they would be disqualified as microorganisms potentially settling the gastrointestinal tract in humans and animals.

It was confirmed that the following bacteria show activity against ureolytic pathogens of the gastrointestinal tract, urinary tract, body surface, and respiratory system:
bacteria L. reuteri DAN080 with bactericidal effect on H. pylori and other pathogens of the gastrointestinal tract by their extracellular metabolism products (FIG. 1).
other lactic acid bacteria constituting the property of the present inventor, which during their growth maintain the capability of releasing alpha-ketoglutarate into the environment as one of their metabolites. Alpha-ketoglutarate in turn, acting locally, in the appropriate concentrations (30 mM), hydrolyses urea present in the environment, thus interfering with the process of colonization by other bacteria ureolytic pathogens, the growth of which is dependent of the pH of the microenvironment, and is impossible in, e.g. acid pH of the stomach.

Within the region of the gastric mucosa this phenomenon covers not only such bacteria as H. pylori, but also Proteus mirabilis, Citrobacter freundii, Klebsiella pneumoniae, Enterobacter cloacae, Staphylococcus aureus, Staphylococcus capitis urealiticum (see: Osaki T et al. Urease-positive bacteria in the stomach induce a false-positive reaction in a urea breath test for diagnosis of Helicobacter pylori infection. J Med Microbiol. 2008; 57:814-9; Brandi G et al. Urease-positive bacteria other than Helicobacter pylori in human gastric juice and mucosa. Am J Gastroenterol. 2006; 101(8):1756-61), which use their own urease for urea decomposition, thus providing themselves with habitation microniches.

### Example 4

### Activity Tests In Vivo

a) Activity of lysozyme: Forty eight 2-month-old Sprague Dawley female rats with a weight of 140-275 g, were fed with feed adequate for the age of the animals, and watered ad libitum. Three days prior to experiment, all animals had catheters inserted in the jugular vein. The study was started by taking blood samples from the rats. Subsequently, the animals were administered intragastrically, by means of a gastric tube, 0.5 ml of the following preparations: suspension of the bacteria L. reuteri DAN080 live and dead cells, chitosan AKG suspension, saline. Hundred twenty min. after the first blood taking the second blood sample was taken from the animals. On the second day, the rats received the same preparations once daily for 7 subsequent days. Twelve rats received live bacteria at a dose of 10⁶ cells suspended in physiological saline. The following 12 animals also received for 8 days, 10⁶ each of thermally killed cells L. reuteri DAN080. To the next 12 rats chitosan AKG suspension was administered, and the fourth group of animals (n=12) were administered intragastrically for 8 days, at each time 0.5 ml physiological saline. On day 8 after the final dose of the preparations administered intragastrically, blood was taken from the animals from the jugular vein. For the second time on the same day, blood was taken from all rats 120 min. after the first blood taking.

Determinations of the lysozyme activity in the blood were performed in the presence of a suspension of Micrococcus lysodeikticus cells of specified density, based on the absorbance value, and comparing this value with the absorbance curve plotted from a number of standard dilutions of crystal lysozyme (Sigma-Aldrich) in PBS and suspension of M lysodeikticus cells of specified density. After 15, 30, 45, 60 min., incubation absorbance was measured at the wave length of 540 nm.

Results: A stimulation of rat immune system was observed by live and thermally killed bacteria L. reuteri DAN080 and by chitosan AKG. FIG. 5 presents the results.

b) The effect of L. reuteri DAN080 metabolites on neurons of the enteric nervous system in swine: Neurons of the enteric nervous system were isolated from the middle section of the small intestine in 3-6 week old piglets (n=5) with a body weight of 15 kg. The tissues were treated with tripsin, type 2 collagenase and protease in order to obtain neuron cultures. The neuron cultures were plated in Neurobasal A medium for neurons, enriched with an additive of fetal bovine serum, or in the presence of neutralized metabolites of L. reuteri DAN080, and twice or four-times concentrated samples of metabolites. The cultures were maintained in an atmosphere of 5% CO₂ at 37° C. for 6 days.

Results: After 6 days of incubation, it was observed in the control samples (neurons cultured on medium) that 53.7±2.7% of cells survived. Considering this fact, the index of neurons was determined which survived in the presence of L. reuteri DAN080 metabolites, with relation to the neurons incubated exclusively on the enriched Neurobasal A medium. The cells of L. reuteri DAN080 isolated from mouse stomach, through their metabolites, do not statistically significantly decrease the survival rate of neurons isolated from the nervous system in piglets. The cells of L. reuteri DAN80 do not cause the degeneration of the nervation (structure) of the gastrointestinal tract. FIG. 5 presents the results.

### Example 5

### The In Vivo Lysozyme Activity Test

Forty eight 2-month-old Sprague Dawley female rats with a weight of 140-275 g, were fed with feed adequate for the age of the animals, and watered ad libitum. Three days prior to experiment, all animals had catheters inserted in the jugular vein. The study was started by taking blood samples from the rats. Subsequently, the animals were administered intragastrically, by means of a gastric tube, 0.5 ml of the following preparations: suspension of the bacteria L. reuteri DAN080—live and dead cells, chitosan AKG suspension, saline. Hundred twenty min. after the first blood taking the second blood sample was taken from the animals. On the second day, the rats received the same preparations once daily for 7 subsequent days. Twelve rats received live bacteria at a dose of 10⁶ cells suspended in physiological saline. The following 12 animals also received for 8 days, 10⁶ each of thermally killed cells L. reuteri DAN080. To the next 12 rats chitosan AKG suspension was administered, and the fourth group of animals (n=12) were administered intragastrically for 8 days, at each time 0.5 ml physiological saline. On day 8 after the final dose of the preparations administered intragastrically, blood was taken from the animals from the jugular vein. For the second time on the same day, blood was taken from all rats 120 min. after the first blood taking.

Determinations of the lysozyme activity in the blood were performed in the presence of a suspension of Micrococcus lysodeikticus cells of specified density, based on the absorbance value, and comparing this value with the absorbance curve plotted from a number of standard dilutions of crystal lysozyme (Sigma-Aldrich) in PBS and suspension of M lysodeikticus cells of specified density. After 15, 30, 45, 60 min., incubation absorbance was measured at the wave length of 540 nm.

Results: A stimulation of rat immune system was observed by live and thermally killed bacteria L. reuteri DAN080 and by chitosan AKG. FIG. 5 presents the results.

### Example 6

The commercially available catheters made of PVC (e.g. Galmed PL), not covered with any protective coating were processed using the methods of surface nanoengineering.

Nanocoating made of PVP was deposited after forming previously an intermediate layer on the basis of chitosan [salts], by a known method of submerging in the solution and air drying at room temperature, and optionally cross-linking by a short-term exposure to the UV radiation and/or to ultrasounds. According to the intended use of the catheter, the thickness of the intermediate layer was regulated by repeating the procedure of applying the first intermediate coating.

The intermediate coating on the basis of chitosan salts was enriched with active substances selected from a group covering thermally inactivated cultures of L. reuteri DAN080, chitosan alpha-ketoglutarate, small dicarboxylic acid, triclosan, silver nanoparticles, and vitamins D and E in the form of nanopowder coated with a protective coating.

Surface nanoengineering allows the manufacturing of an intermediate coating of the thickness of approximately 50,000 C—C bonds (10 nm).

The PVP polymer nanocoating of the indicated thickness, in the environment of physiological pH, is totally dissolved during usage. Gradual dissolving of the PVP layer reveals gradually the intermediate coating, wherefrom gradually active substances are released by diffusion. A single intermediate coating maintains its durability for the period up to one week, preventing the formation of biofilm, development of irritations and inflammatory states.

After 7 days of the catheter presence in the bodies of patients subjected to the urinary tract catheterization, in healthy volunteers no undesirable responses and reactions were noted.

Present studies concerning the catheters coated with hydrogel nanocoating show, for the first time, promising results with respect to the possibility of reduction of the frequency of CAUTI occurrence.

Even when a catheter is manufactured from non-invasive materials, these materials are recognized as foreign bodies by the cells of the immune system. The catheter according to the invention with currently disclosed external nanocoatings, in contact with the epithelial cells of a patient, does not exert any cytotoxic effect on the epithelium. Nanoparticles used in the study are not recognized as dangerous by the host cells. When evaluating a potential biological effect of the new material, consisting of exposing the antibacterial nanocoating to the liver tissues, the analysis showed a lack of hepatocytes reaction to the insertion of the catheter into the catchment area of the portal vein. A considerable improvement of the catheter was proposed with respect to the materials used. Due to the natural antibacterial coating, the new generation of catheters coated with hydrogel is characterized by lower contact friction, and reduces the urinary tract inflammatory processes and infections in a way similar to traditional antibiotic therapy. The catheter is cheaper and more beneficial for patients, as compared to traditional catheters.

### Example 7

The kit according to the invention comprises a catheter as described in Example 6 above, a vial with water for injection (sterile), and a stress-reducing agent for oral administration, in the form of live or heat inactivated L. reuteri DAN080 cultures, at a dose of 10⁶ cells, for everyday administration for at least the period of catheterization. Depending on the patient status, the physician in charge may order administration of a stress-reducing agent also during the period preceding catheterization. The oral administration of the stress-reducing agent is recommended at 8 hours before the catheterization, or the administration directly into the body cavity at 15 min. before the catheterization.

### Example 8

Topical application of L. reuteri DAN080 culture has been tested for the prevention of superficial skin and burn wound infections. Studies involving the use of L. reuteri DAN080 culture (immobilized from calcium chitosan or calcium alginate films) investigated the antibacterial activity of these films in a burn wound model in rats. A multiresistant clinical isolate, ureasepositive Pseudomonas aeruginosa, served as the indicator strain. Films incorporating L. reuteri DAN080 culture (equilibrated to cell concentrations of 10⁸ CFU/mL) caused a reduction of 5-6 log(10) in P. aeruginosa in the model burn wounds. Wound dressings containing immobilized L. reuteri DAN080 culture in freeze-dried calcium chitosan or alginate films remained viable for six months of storage at 4° C. This indicates that L. reuteri DAN080 culture and/or its by-products express potential therapeutic activities for the local treatment of P. aeruginosa burn infections.

The burn-mouse model of (Rumbaugh K P et al. Contribution of quorum sensing to the virulence of Pseudomonas aeruginosa in burn wound infections. Infect Immun 1999; 67:5854-5862), was used in the study. Anaesthetised mice whose backs were shaved, were placed in a water bath at a temp. 90 C for 10 s to burn the neck surface. One group of mice (B group) randomly selected, obtained a PBS injection directly under the burn and the second group was infected with 100 ul of 200-300 CFU of P. aeruginosa (BPs group), half of the mice from the second group was treated with L. reuteri DAN080 culture on days 3, 4, 5, 7 and 9 after the initial infection with L. reuteri DAN080 culture (equilbrate 100 ul of 10⁵ DAN080 cells grown in MRS broth (BPs+DAN080 group). On days 5, 10, 15 after the initial infection, mice were sacrificed and blood samples, skin, connective tissue and muscle from the burn area were collected and processed. Histological studies showed that on day 5 edema, vascular congestion and necrotic areas containing inflammatory infiltrates developed, these infiltrates were larger in groups BPs and BPS+DAN080 than in group B. On day 10 the wound repair process was significantly advanced in group B compared to the other groups. In group BPs+DAN080 the necrosis area was smaller and the inflammatory infiltrates more diffuse than in the mice from BPs group. At day 15, 62% of the mice in group BPs+DAN080 showed a clearance of bacteria, compared to 38% in group BPs.

1. Lactobacillus reuteri DAN080 strain with deposit number DSM 15693 for use in medicine.

2. Lactobacillus reuteri DAN080 strain with deposit number DSM 15693 as claimed in claim 1, in form of a culture, a partially inactivated culture; liquid, concentrated and dried supernatant of L. reuteri DAN080 with deposit number DSM 15693 culture, for use in medicine as therapeutic and prophylactic agent, especially as an antimicrobial agent, in prophylaxis and treatment of medical conditions developing as a result of infections caused by bacteria, fungi, and other pathogens of the gastrointestinal tract, body surface, and other systems, such as urogenital system, respiratory system, in vertebrates or as a therapeutic and/or prophylactic agent for the treatment and prevention of development of gout (podagra) and/or for increasing the activity of lysozyme in an organism of vertebrate, especially human, other mammal or bird.

3. Lactobacillus reuteri DAN080 strain with deposit number DSM 15693 as claimed in claim 1, in form selected from the group comprising the whole L. reuteri DAN080 strain with deposit number DSM 15693 culture; liquid, concentrated and dried supernatant obtained from L. reuteri DAN080 strain with deposit number DSM 15693 culture and from mixed bacterial cultures containing L. reuteri DAN080 strain with deposit number DSM 15693; and liquid, concentrated and dried supernatants obtained from the cultures of prokaryotic and eukaryotic recombinants, and the whole cultures of prokaryotic and eukaryotic recombinants, in which and/or from which genes are utilized, which genes provide specific modulatory, inhibitory, and homeostatic activity with respect to H. pylori and other bacteria; proteins/oligopeptides/peptides of molecular weight of approximately: 150 and/or 141 and/or 115 and/or 95 and/or 90 and/or 86 and/or 83 and/or 77 and/or 71 and/or 63 and/or 59 and/or 56 and/or 49 and/or 46 and/or 43 and/or 39 and/or 34 and/or 32 and/or 30 and/or 22 kD, or lower, purified/isolated from liquid, concentrated, dried supernatants, obtained from L. reuteri DAN080 strain with deposit number DSM 15693; and purified/isolated from the whole L. reuteri DAN080 strain with deposit number DSM 15693 cultures; and from other mixed bacterial cultures, which included L. reuteri DAN080 strain with deposit number DSM 15693; and from purified or isolated cultures of prokaryotic and eukaryotic recombinants, in which and/or from which genes are utilized, which genes provide specific modulatory, inhibitory, and homeostatic activity with respect to H. pylori and other bacteria; or their mixtures, for use in medicine as an antimicrobial agent in prophylaxis and treatment of medical conditions developing as a result of infections caused by bacteria, fungi, and other pathogens of the gastrointestinal tract, body surface, and other systems, such as urogenital system, respiratory system, in vertebrates.

4. Lactobacillus reuteri DAN080 strain with deposit number DSM 15693 as claimed in claim 1, for use as an antimicrobial agent, as a therapeutic and/or prophylactic agent for the treatment and prevention of development of gout (podagra) and/or for increasing the activity of lysozyme in an organism of vertebrate, wherein the vertebrate is a human individual.

5. Lactobacillus reuteri DAN080 strain with deposit number DSM 15693 as claimed in claim 1, for use as an antimicrobial agent, as a therapeutic and/or prophylactic agent for the treatment and prevention of development of gout (podagra) and/or for increasing the activity of lysozyme in an organism of vertebrate, wherein the vertebrate is a domestic animal, pet, animal involved in sport, broiler, layer hen, mouse, rat, guinea pig, rabbit and other laboratory animals, including primates, independent of its age.

6. Lactobacillus reuteri DAN080 strain with deposit number DSM 15693 as claimed in claim 1, for use as an antimicrobial agent, wherein the microorganism is a pathogenic bacterium or fungus.

7. Lactobacillus reuteri DAN080 strain with deposit number DSM 15693 as claimed in claim 5, for use as an antimicrobial agent, wherein the pathogenic bacterium is Helicobacter pylori.

8. Lactobacillus reuteri DAN080 strain with deposit number DSM 15693 as claimed in claim 1, for manufacturing a composition for modulation of function of stomach, intestine, and GIT in vertebrate in need of such a treatment, including human, other mammal and bird, or for treatment and prevention of development of gout (podagra) and/or for increasing the activity of lysozyme in an organism of the vertebrate, comprising said Lactobacillus reuteri DAN080 strain with deposit number DSM 15693 in an effective amount, providing achieving the desired preventive or therapeutic result.

9. The use according to claim 8, wherein the composition is designed for killing, inhibiting, regulating, and preventing the growth of H. pylori and other microorganisms, or for treatment and prevention of development of gout (podagra) and/or for increasing the activity of lysozyme in an organism of the vertebrate, and for the administration in an effective amount and at a appropriate rate, necessary for obtaining the desired preventive or therapeutic result.

10. The use according to claim 8, wherein the composition is designed for the treatment, alleviation, or prophylaxis of GIT disorders, gastritis, gastric ulcer, duodena] ulcer, gastric cancer, duodenal cancer or for treatment and prevention of development of gout (podagra) and/or for increasing the activity of lysozyme in an organism of the individuals in need of such a treatment and being vertebrates, including mammals and birds.

11. The use according to the preceding claim 10, wherein the composition is designed for the treatment, alleviation, or prophylaxis of diarrhea.

12. The use according to the preceding claim 11, wherein treatment, alleviation, or prophylaxis of diarrhea includes elimination or stabilization of H. pylori growth.

13. The use according to the preceding claim 10, wherein the composition is designed for the for treatment and prevention of development of gout (podagra) and/or for increasing the activity of lysozyme in an organism of the vertebrate.

14. The use according to claim 8, wherein the composition is a pharmaceutical composition optionally containing other biologically active substances, such as vitamins, especially D and E, in particular in a nanoform, at preventive or therapeutic doses, pharmaceutically acceptable carriers and/or other additives, as well as antimicrobial chitosan salts, especially alpha-ketoglutarate, citrate and lactate.

15. The use according to claim 14, wherein the pharmaceutical composition is in solid form, divided into single doses containing a therapeutically effective amount of said Lactobacillus reuteri DAN080 strain with deposit number DSM 15693, in amount from 0.001 to 0.2 g/kg of body weight per day.

16. The use according to claim 15, wherein the pharmaceutical composition is in the form of tablets or capsules.

17. The use according to claim 14, wherein the pharmaceutical composition is in liquid form, divided into single doses containing a therapeutically effective amount of said Lactobacillus reuteri DAN080 strain with deposit number DSM 15693, in amount from 0.001 to 0.2 g/kg of body weight per day.

18. The use according to claim 17, wherein the pharmaceutical composition is in liquid form designed for use as aerosol, cataplasm or moist compress.

19. The use according to claim 8, wherein the composition is a dietary supplement, food or feed additive.

20. The use according to claim 19, wherein the dietary supplement, food or feed additive is in a solid form and/or in the form of a beverage.

21. The use according to claim 19, wherein the therapeutically effective amount ranges from 0.001 to 0.2 g/kg of body weight per day.

22. A catheter for insertion into body vessels, ducts and/or cavities, for use in prophylaxis, diagnostics and medicine, both human and veterinary, made of plastic and coated with a protective lubricant layer, characterized in that it has an outer nanocoating of a biocompatible polymer capable of forming gel with water, permanently attached to said plastic either directly or through a nanocoating of a polymer chemically bonded to the catheter material and having antibacterial properties, wherein at least one of the nanocoatings comprises an addition of extracellular metabolites secreted by Lactobacillus reuteri DAN080 strain with deposit number DSM 15693, said metabolites having antimicrobial and anti-inflammatory activity, as well as an optional addition of vitamin D in form of nanoparticles.

23. The catheter according to claim 22, characterized in that the biocompatible polymer is polyvinyl pirrolidone, and the nanocoating made of this polymer has a thickness of about 50,000 C—C bonds (10 nm).

24. The catheter according to claim 22, characterized in that the polymer having antibacterial properties is a salt of chitosan and small organic acid, preferably alpha-ketoglutaric acid.

25. The catheter according to claim 22, characterized in that additional active agents selected from the group comprising chitosan alpha-ketoglutarate, chitosan citrate, chitosan lactate exhibiting antimicrobial and anti-inflammatory activity, small dicarboxilic acid, silver nanoparticles and vitamins D and E in the form of nanopowder coated with a protective coating, and the combinations thereof, are dispersed in the nanocoating made of biocompatible polymer and/or in the polymer having antibacterial properties.

26. A catheterization kit comprising the catheter according to claim 22, and a vial with water for injection (sterile), and a stress-reducing agent for oral administration in the form of live or thermally inactivated Lactobacillus reuteri DAN080 strain with deposit number DSM 15693 cultures at a dose of 10⁶ cells, for daily administration for the period of catheterization and/or at 8 hours prior to catheterization, or directly to the body cavity at 15 min. prior to catheterization.

27. The kit according to claim 26, characterized in that the water container is fixed at the tip of the catheter, and has a partition dividing water from the catheter, which partition is destroyed by the rotation of the container against the catheter when the catheter protrudes outside from the package.

28. Lactobacillus reuteri DAN080 strain with deposit number DSM 15693 as claimed in claim 1, for use in manufacture of hygienic articles in form of a dressing, diapers, tampons, bandages, bandaids, sanitary pads, sanitary napkins with wings, panty liners, cosmetic pads, wraps for animals for night and day use and other personal hygiene articles, as antimicrobial agent.

29. Use according to claim 28, wherein the article is designed as a plastic protector or fire blanket for rescue units, including fire rescues, intended for patients with severe bums or road traffic accident victims with extensive bodily injuries.

30. Use according to claim 29, wherein the plastic protector or fire blanket is coated with a nanolayer comprising Lactobacillus reuteri DAN080 strain with deposit number DSM 15693.

31. Use according to claim 30, wherein Lactobacillus reuteri DAN080 strain with deposit number DSM 15693 is released in contact with the burned or injured patient's body in antimicrobially effective amount.
